SEQUENCE LISTING (8 sequences)

SEQ ID NO: 1 - DNA, 21 bp, Artificial Sequence
  oligonucleotide primer
caacactgccaacgtccagat

SEQ ID NO: 2 - DNA, 21 bp, Artificial Sequence
  oligonucleotide primer
ctgcttcgtccagataggcaa

SEQ ID NO: 3 - DNA, 21 bp, Artificial Sequence
  oligonucleotide primer
tgaaaccacctctgcattcca

SEQ ID NO: 4 - DNA, 21 bp, Artificial Sequence
  oligonucleotide primer
gacgcctcgccttcttgaaat

SEQ ID NO: 5 - DNA, 21 bp, Artificial Sequence
  oligonucleotide primer
tgctcaaaggctacgactgga

SEQ ID NO: 6 - DNA, 19 bp, Artificial Sequence
  oligonucleotide primer
ttgacgtgcggcttgttct

SEQ ID NO: 7 - DNA, 19 bp, Artificial Sequence
  oligonucleotide primer
atggggaaggtgaaggtcg

SEQ ID NO: 8 - DNA, 20 bp, Artificial Sequence
  oligonucleotide primer
taaaagcagccctggtgacc

(19) United States
(12) Patent Application Publication
(10) Pub. No.: US 2019/0030211 A1
(43) Pub. Date: Jan. 31, 2019

(54) HYDROGEL SCAFFOLD FOR THREE DIMENSIONAL CELL CULTURE

(71) Applicant: The Regents of the University of California, Oakland, CA (US)

(72) Inventors: Jin Nam, Irvine, CA (US); Alexander Brunelle, Riverside, CA (US)

(21) Appl. No.: 16/050,510

(22) Filed: Jul. 31, 2018

Related U.S. Application Data
(60) Provisional application No. 62/539,429, filed on Jul. 31, 2017.

(51) Int. Cl. A61L 27/44; C12M 3/00; C12N 5/0775

(52) U.S. Cl. CPC A61L 27/44; C12M 21/08; A61L 27/3882; C12N 5/0665; C12N 5/0668

(57) ABSTRACT

A composite microfiber including an electrospun blend of a thermosensitive hydrogel and a biodegradable polymer, a scaffold including a plurality of the composite microfibers, optionally including cells cultured in the scaffold, and methods of making an engineered tissue including seeding cells within a plurality of composite microfibers.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### STATEMENT REGARDING SEQUENCE LISTING

The present application is being filed along with a Sequence Listing in electronic format. The Sequence Listing is provided as a file entitled 28700098 1.TXT, created Jul. 31, 2018, which is 1.85 Kb in size. The information in the electronic format of the Sequence Listing is incorporated herein by reference in its entirety.

### FIELD OF THE INVENTION

A scaffolding system that includes a thermosensitive hydrogel and a biodegradable polymer blended into a composite, electrospun microfibrous structure. Also disclosed are related methods of making engineered tissues.

### BACKGROUND

Senescence-related degenerative diseases, such as osteoarthritis, osteoporosis and various vascular disorders, have become prevalent due to the exponential growth of the aging population [1]. Tissue engineering approaches, especially those using stem cells, present a viable means to produce tissue replacements to remedy such diseases while overcoming several issues associated with allografts, such as shortage in donor organs and immuno-incompatibility. In addition to their potential for in-vivo implantation, engineered tissues also provide valuable in-vitro tissue models as platforms to develop therapeutic interventions including drug discovery/testing [2-4]. Depending on the complexity and dimensions of the assembly, two different approaches are typically utilized to create biomimetic in-vitro tissues. A scaffold-less approach utilizes individual cells or a patch of cells to assemble a structure resembling native tissue [5]. This process offers the ability to control each layer individually as they are added, enabling complex composition and spatial distribution of cells with various phenotypes. In contrast, a scaffold-based approach simplifies the tissue morphogenesis process by using a physical structure as a cell culture substrate, allowing for the generation of engineered tissues with relatively large dimensions. The consequence for its simplicity is the need for a well-designed structure to guide appropriate cellular behaviors such as stem cell self-renewal, differentiation and maturation.

Hydrogels are one of the most popular scaffolding systems due to their capability to encapsulate cells in a 3D physiological-like microenvironment [6, 7]. Many different types of hydrogels have been developed through chemical modification and functionalization to direct stem cell behaviors for enhanced morphogenesis of various tissues, such as angiogenesis [8], neurogenesis/myogenesis [9], osteogenesis/adipogenesis [10], and dermal tissue formation [11]. Notably, several hydrogel systems have demonstrated an excellent ability to enhance chondrogenesis of stem cells due to their hydrated fibrous nanostructure providing a cellular environment similar to native cartilage [12-16]. However, a major drawback of using hydrogel scaffolds is the necessity for multi-step synthesis processes for cell/scaffold constructs with defined dimensions, limiting their off-the-shelf usage. The process generally requires dissolution of a hydrogel precursor polymer before mixing with cell-containing solution, followed by a gelation step via chemo-, photo- or thermo-sensitive crosslinking inside a mold to form cell-encapsulating hydrogels with a defined shape [17]. The elimination of these multi-step processes by increasing dissolution rate in cell-containing media and enabling in-situ polymerization without the need for a mold, would facilitate the use of hydrogel scaffolds in surgical procedures.

In this regard, electrospinning provides a means to produce a fibrous network exhibiting a very high surface area for faster dissolution. The non-woven mesh of fibers can be produced with diameters ranging from a few nanometers to several micrometers, by electrically pulling a polymer solution with high electric potentials [18]. Any polymer solution with the appropriate viscosity, conductivity and surface tension can be electrospun for a variety of applications. A few hydrogels, such as poly (ethylene glycol) (PEG) [19] and poly(N-isopropylacrylamide) (PNIPAAm) [20, 21], have been electrospun to create fibrous scaffolds. However, these electrospun hydrogel scaffolds collapse upon cell seeding, unable to maintain their macro-scale structures.

A composite structure consisting of fibers that contain PEG-PNIPAAm and PCL is reported by Hui Yu et al. (2016 “Fabrication of core/sheath PCL/PEG-PNIPAAm fibers as thermosensitive release carriers by a new technique combining blend electrospinning and ultraviolet-induced graft polymerization” Materials Letters 164: 505-508). However, in Yu et al., a PCL fiber core is initially made by electrospinning and then PEG-PNIPAAm is grafted onto the PCL fiber cores by UV-induced graft polymerization. Yu et al. refers in the introduction section to electrospinning methods that use multiple co-flows. However, Yu et al. discourages blending of polymers prior to electrospinning, stating that the previous attempts produced fibers with unsatisfactory drug distribution.

Li et al. (2005 “Electrospun protein fibers as matrices for tissue engineering” Biomaterials 26: 5999-6008) teaches electrospinning of protein fibers as matrices for tissue engineering, but there is no basis to form such fibers from blended PEG-PNIPAAm and PCL.

Scaffaro (2017 “Nanocarbons in Electrospun Polymeric Nanomats for Tissue Engineering: A Review” Polymers 9: 76) teaches the use of electrospun fibers for use in tissue engineering scaffolds, but the reference provides no basis for fibers made from blended PEG-PNIPAAm and PCL.

### SUMMARY OF THE INVENTION

Hydrogels have shown great potential for cartilage tissue engineering applications due to their capability to encapsulate cells within biomimetic, 3-dimensional (3D) microenvironments. However, the multi-step fabrication process that is necessary to produce cell/scaffold constructs with defined dimensions, limits their off-the-shelf translational usage. We have developed a hybrid scaffolding system which combines a thermosensitive hydrogel, poly(ethylene glycol)-poly(N-isopropylacrylamide) (PEG-PNIPAAm), with a biodegradable polymer, poly(c-caprolactone) (PCL), into a composite, electrospun microfibrous structure. A judicious optimization of material composition and electrospinning process produced a structurally self-supporting hybrid scaffold. The reverse thermosensitivity of PEG-PNIPAAm allowed its dissolution/hydration upon cell seeding within a network of PCL microfibers while maintaining the overall scaffold shape at room temperature. A subsequent temperature elevation to 37° C. induced the hydrogel's phase transition to a gel state, effectively encapsulating cells in a 3D hydrogel without the use of a mold. We demonstrated that the hybrid scaffold enhanced chondrogenic differentiation of human mesenchymal stem cells (hMSCs) based on chondrocytic gene and protein expression, which resulted in superior viscoelastic properties of the cell/scaffold constructs. The hybrid scaffold enables a facile, single-step cell seeding process to inoculate cells within a 3D hydrogel with the potential for cartilage tissue engineering.

Some embodiments relate to a composite microfiber comprising an electrospun blend of a thermosensitive hydrogel and a biodegradable polymer.

In some embodiments, the thermosensitive hydrogel is poly(ethylene glycol)-poly(N-isopropylacrylamide) (PEG-PNIPAAm) and wherein the biodegradable polymer is poly(ϵ-caprolactone) (PCL), wherein the PEG-PNIPAAm and PCL are blended throughout the fiber.

In some embodiments, the biodegradable polymer is natural biodegradable polymer.

In some embodiments, the diameter of the fibers is in the range of 1-50 microns.

In preferred embodiments, the diameter of the fibers is in the range of 9-13 microns, with a particularly preferred diameter of 11 microns.

In some embodiments, the ratio of thermosensitive hydrogel: biodegradable polymer is 65:35.

Some embodiments relate to a scaffold comprising a plurality of composite microfibers as disclosed herein.

In some embodiments, the scaffold has a thickness of about 2.5 mm.

In some embodiments, the scaffold further comprises cells cultured within the scaffold.

In some embodiments, the scaffold has a compressive modulus of between 40-80 kPa.

In some embodiments, the scaffold has a compressive modulus of about 60 kPa.

In some embodiments, the scaffold and cells form a shape.

Some embodiments relate to A method of making an engineered tissue, the method including:

obtaining a plurality of composite microfibers as disclosed herein;

seeding cells within the plurality of composite microfibers, while the plurality of composite microfibers is in a dry state and the cells are dispersed in a solution, and inducing a phase transition of the thermosensitive hydrogel to a gel state upon elevating the temperature of the plurality of composite microfibers and cells, thereby encapsulating the cells in a 3D hydrogel scaffold that forms the engineered tissue.

Some embodiments of the method further comprise implanting in a subject the plurality of composite microfibers seeded with the cells, wherein a body temperature of the subject causes the elevating of the temperature of the plurality of composite microfibers and cells, which causes the hydrogel to undergo the phase transition to the gel state.

In some embodiments, the engineered tissue is a soft tissue, a hard tissue, or a combination of soft and hard tissues selected from the group consisting of bone and cartilage.

In some embodiments the cells include mesenchymal stem cells, and the mesenchymal stem cells are cultured within the scaffold under conditions that promote chondrogenic differentiation, thereby producing cartilage tissue.

In some embodiments, the mesenchymal stem cells are human mesenchymal stem cells (hMSCs).

In some embodiments, the engineered tissue is a soft tissue.

In some embodiments, the soft tissue is selected from the group consisting of skin, a vein, an artery and an organ.

### BRIEF DESCRIPTION OF THE DRAWINGS

FIG. 1. Schematic of 3D cell encapsulation in mold-less hydrogel using electrospun hybrid scaffolds. Thermosensitive PEG-PNIPAAm composited with PCL, was electrospun to produce thick (˜2.5 mm) hybrid scaffolds composed of microfibers. Upon the inoculation of cell-suspended media solution at room temperature (RT), large pores allow uniform cell infiltration throughout the thickness of the scaffolds, while dissolving the PEG-PNIPAAm component from the microfiber. Subsequent increase in temperature to 37° C. induces the gelation of the dissolved PEG-PNIPAAm to encapsulate the seeded cells by the hydrogel in 3D.

FIG. 2. Chemical and functional characterization of PEG-PNIPAAm. The chemical structure of the synthesized PEG-PNIPAAm was characterized by (a) FTIR and (b) H NMR. The FTIR characteristic absorbance peaks of PEG-DMA (blue) and NIPAAm (red) monomers were compared to those of the synthesized PEG-PNIPAAm (green), showing that the characteristic peaks of PEG-DMA (1: epoxy group C-H stretching, 2: C-H bending,) and NIPAAm (3: N—H stretching, 4: amide groups C═O and N—H bending, 5:isopropyl group splitting) are present in PEG-PNIPAAm. The H NMR characteristic peaks of PEG-DMA (1: PEG chain proton) and NIPAAm (2: C—H proton, 3: methylene proton) further validate the synthesis of PEG-PNIPAAm. An example of temperature-sensitive gelation behaviors of PEG-PNIPAAm at a solution concentration of 5 wt. % as compared to a control cell culture medium (0 wt. %) is shown at (c) room temperature (23° C.) and (d) 37° C. (e) The lower critical solution temperature (LCST) was determined by subjecting different concentrations of PEG-PNIPAAm to a temperature sweep.

FIG. 3. Concentration-dependent cell viability in PEG-PNIPAAm hydrogels. Human MSCs were cultured in PEG-PNIPAAm hydrogels with different concentration of (a) 5 wt. %, (b) 7.5 wt. % or (c) 10 wt. % for 24 hrs, and subjected to a live-dead cell assay, showing live (green) and dead (red) cells under fluorescence microscopy. (d) Cell viability was quantified from the fluorescent images (scale bar: 100 μm). * denotes statistical significance of p<0.05. ns denotes no statistical significance (n=45).

FIG. 4. Morphological characterization of as-spun and hydrated PEG-PNIPAAm:PCL hybrid electrospun scaffolds. Microstructure of (a, b, c, d) as-spun or (e, f, g, h) hydrated electrospun scaffolds ((a, e) pure PCL, (b, f) 25% PEG-PNIPAAm:PCL blends, (c, g) 50% PEG-PNIPAAm:PCL blends, (d, h) 65% PEG-PNIPAAm:PCL blends. The hydrated samples were lyophilized before imaging (scale bar: 50 μm).

FIG. 5. Morphological characterization of PCL backbone in the hybrid electrospun scaffolds. (a) 65% PEG-PNIPAAm scaffolds were washed with PBS overnight at 23° C. to remove the hydrogel component from the scaffold (scale bar: 50 μm), revealing (b) pit formation in the PCL fibers without losing the overall microfibrous structure (scale bar: 5 μm).

FIG. 6. Mechanical characterization of PEG-PNIPAAm:PCL hybrid electrospun scaffolds. PEG-PNIPAAm:PCL scaffolds with various blending ratios (25%, 50% and 65%) were subjected to sequential mechanical testing (1) as-spun at 23° C. (Dry 23° C.), (2) after hydrated at 37° C. (Wet 37° C.), (3) followed by cooled down to 23° C. (Wet 23° C.). (a) The dynamic compressive moduli were decomposed to (b) elastic (G′) and (c) viscoelastic moduli (G″) at the different hydration and temperature conditions. Pure PCL scaffolds (indicated as 0%) and pure PEG-PNIPAAm at 37° C. (indicated by red dotted line) were used as controls. *, † and ‡ denote statistical significance (p<0.05) with respect to pure PCL control (0%) scaffolds within the same environmental condition, the same scaffold type in the Dry 23° C. condition, and pure PEG-PNIPAAm hydrogel at 37° C., respectively (n=6).

FIG. 7. Localization of the inoculated cells in PEG-PNIPAAm:PCL hybrid electrospun scaffolds. Human MSCs were seeded into (a) 25%, (b) 50% and (c) 65% PEG-PNIPAAm:PCL hybrid scaffolds having a thickness of approximately 2.5 mm. The cross-sectional images show that cells seeded in the 65% scaffolds are evenly distributed throughout the thickness of scaffolds while cells seeded in the 25% and 50% scaffolds exhibited sedimentation toward the bottom. Dashed lines outline the top and bottom of the cell-seeded scaffolds. Cells are visualized by DAPI nuclear staining (scale bar: 500 μm).

FIG. 8. Enhanced chondrogenic differentiation of human MSCs cultured in the PEG-PNIPAAm:PCL hybrid scaffolds. Chondrogenic gene expression of (a) COL2A1, (b) ACAN, and (c) SOX9 was upregulated in the cells cultured in the 65% PEG-PNIPAAm hybrid scaffolds (65%) or pure PEG-PNIPAAm hydrogels (100%) for two weeks compared to that of the cells cultured on the tissue culture polystyrene as a negative control (Neg) and in the pure PCL scaffolds (0%). * and ** denote statistical significance of p<0.05 and p<0.01, respectively (n=6). (d) Histochemical staining with Safranin-O and Alcian blue shows enhanced production/deposition of chondrogenic ECM in the hybrid scaffolds as compared to pure PCL (scale bar: 100 μm).

FIG. 9. Mechanical characterization of cell/scaffold constructs. Two-week culture of hMSCs in the 65% PEG-PNIPAAm hybrid scaffolds (65%) resulted in a superior (a) dynamic compressive, (b) elastic (G′) and (c) viscoelastic (G″) moduli over the pure PCL control (0%) or the pure PEG-PNIPAAm hydrogel control (100%). * and ** denote statistical significance of p<0.05 and p<0.01, respectively (n=6).

FIG. 10. Lower critical solution temperature characterization depending on the total pre-cursor concentration during PEG-PNIPAAm synthesis. (a) various total pre-cursor concentrations (NIPAAm and PEG-DMA in methanol) of 5 wt. % (teal), 7.5 wt. % (orange), 10 wt. % (purple) and 12.5 wt. % (green) with a fixed NIPAAm:PEG-DMA ratio of 9:1 were used to synthesize PEG-PNIPAAm with different gelation behaviors determined by subject different concentrations of the synthesized polymer to a temperature sweep. Reactions above 12.5 wt. % resulted in incomplete free-radical polymerization. (b) The lowest concentration to form a hydrogel at any temperature (critical concentration) was inversely dependent on the reaction pre-cursor concentrations during synthesis.

FIG. 11. 25% PEG-PNIPAAm Hybrid Electrospun Fiber Optimization. Based on the DOE in Table 2, the electrospinning parameters, overall polymer concentration and collector distance, were optimized to produce microfibers having an average fiber diameter of approximately 11 μm with a typical cylindrical morphology. First, maximum (+) and minimum (−) electrospinning conditions at which cylindrical fibers were achievable, were determined for both weight percentage (wt. %) and distance (D). Within these limits, five conditions were electrospun: SEM images of (a) −wt. %, −D; (b) −wt. %, +D; (c) median (mid) wt. %, mid D; (d) +wt. %, −D; (e) +wt. %, +D (scale bar: 50 μm). Average fiber diameters were digitally measured using ImageJ software (n=100). (f) Using the DOE, the magnitude and direction of the effect of the electrospinning variables on fiber diameter were determined. (g) Three-dimensional plot of polymer solution concentration vs. collector distance vs. electrospun fiber diameter showing optimized (red) and non-optimized (blue) electrospinning conditions.

FIG. 12. 50% PEG-PNIPAAm Hybrid Electrospun Fiber Optimization. Based on the DOE in Table 2 and electrospinning limits to produce a typical cylindrical fiber morphology, five conditions were electrospun: SEM images of (a) −wt. %, −D; (b) −wt. %, +D; (c) median (mid) wt. %, mid D; (d) +wt. %, −D; (e) +wt. %, +D (scale bar: 50 μm). Average fiber diameters were digitally measured using Image J software (n=100). (f) Using the DOE, the magnitude and direction of the effect of the electrospinning variables on fiber diameter were determined. (g) Three-dimensional plot of polymer solution concentration vs. collector distance vs. electrospun fiber diameter showing optimized (red) and non-optimized (blue) electrospinning conditions.

FIG. 13. 75% PEG-PNIPAAm Hybrid Electrospun Fiber Optimization. Based on the DOE in Table 2 and electrospinning limits to produce a typical cylindrical fiber morphology, five conditions were electrospun: SEM images of (a) −wt. %, −D; (b) −wt. %, +D; (c) median (mid) wt. %, mid D; (d) +wt. %, −D; (e) +wt. %, +D (scale bar: 50 μm). Average fiber diameters were digitally measured using Image J software (n=100). (f) Using the DOE, the magnitude and direction of the effect of the electrospinning variables on fiber diameter were determined. (g) Three-dimensional plot of polymer solution concentration vs. collector distance vs. electrospun fiber diameter showing the electrospinning conditions tested. All conditions produced non-optimized (blue) fiber diameters. (h) When distance was decreased further than the minimum, fiber mats became fused. (i) When wt. % was increased from the maximum, fiber morphologies became flat and limited the pore diameter of the scaffolds.

FIG. 14. 65% PEG-PNIPAAm Hybrid Electrospun Fiber Optimization. To replace the 75% condition, 65% PEG-PNIPAAm scaffolds were electrospun under a similar five conditions to 25% and 50% PEG-PNIPAAm fibers: SEM images of (a) −wt. %, −D; (b) −wt. %, +D; (c) median (mid) wt. %, mid D; (d) +wt. %, −D; (e) +wt. %, +D (scale bar: 50 μm). Average fiber diameters were digitally measured using Image J software (n=100). (f) Using the DOE, the magnitude and direction of the effect of the electrospinning variables on fiber diameter were determined. (g) Three-dimensional plot of polymer solution concentration vs. collector distance vs. electrospun fiber diameter showing optimized (red) and non-optimized (blue) electrospinning conditions.

FIG. 15. Representative stress-strain curves of various scaffolds under dynamic compressive forces (a) A representative stress (solid black)-strain (dashed red) curve of the pure PCL scaffold at 23° C. Hybrid scaffolds of 65% PEG-PNIPAAm blend were subjected to mechanical testing (b) as-synthesized at 23° C., (c) after gelled at 37° C. and (d) after cooled to 23° C.

FIG. 16. A picture of cell-laden pure PEG-PNIPAAm casted in a PDMS mold, indicated by arrow.

FIG. 17. A representative fluorescence image showing localization of seeded hMSCs within the 65% hybrid scaffolds. The cells visualized by Calcein-AM staining were located in the macropores (marked by arrows) as well as on the PCL fibers, which are detectable due to autofluorescence.

FIG. 18. The inflammatory gene expression of hMSCs cultured in the 65% hybrid scaffolds. hMSCs were cultured in the hybrid scaffold for 24 hours, and their gene expression for inflammatory markers including COX2, IL6, IL10 and IDO1 was compared to that of TCPS-cultured cells. No statistical difference was detected in these genes.

### DETAILED DESCRIPTION

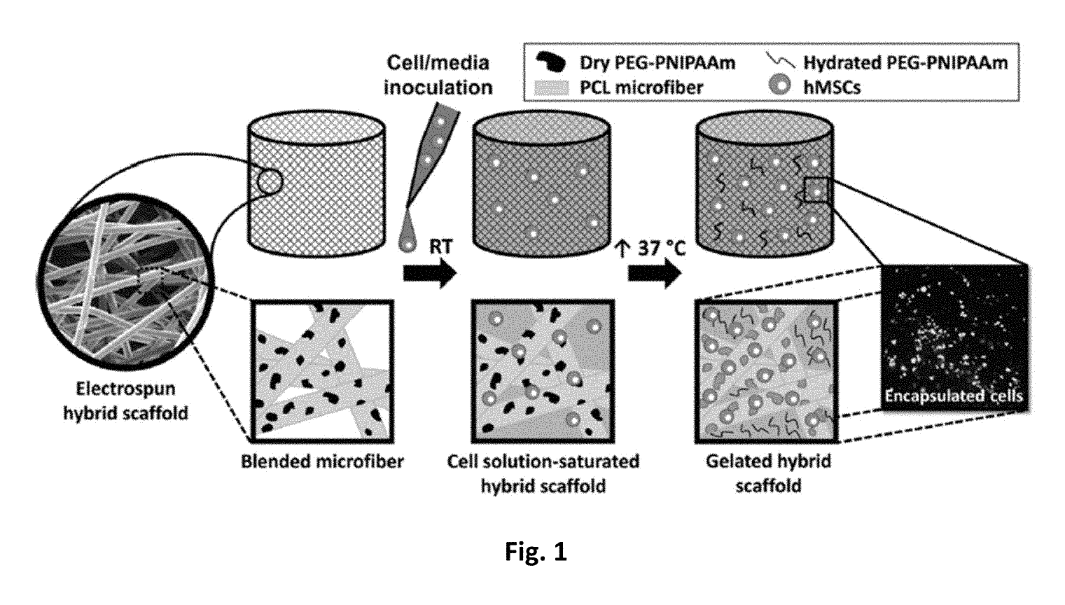

We developed a novel electrospun hybrid scaffold which integrates a thermosensitive hydrogel, PEG-PNIPAAm, with a biodegradable polymer, poly(ϵ-caprolactone) (PCL). We demonstrate that the thermosensitive hydrogel component of the hybrid scaffold quickly dissolves and fills the pores within the structure to encapsulate seeded cells, while PCL provides overall structural integrity, resulting in superior mechanical properties of cell/scaffold constructs as compared to scaffolds of the pure forms of its individual constitutive components (FIG. 1). The hybrid scaffold of cell-conforming hydrogel within electrospun fibrous network enhanced chondrogenic differentiation of human mesenchymal stem cells (hMSCs). Our novel scaffold design provides a facile means to inoculate cells in 3D hydrogel with a mold-less, single step cell seeding process, potentially applicable for various tissue engineering applications.

### Thermosensitive Hydrogels

Thermosensitive hydrogels can advantageously be used in cell encapsulation and tissue engineering applications. Thermosensitive hydrogels include chitosan and related derivatives, poly(N-isopropylacrylamide)-based (PNIPAAM) copolymers, poly(ethylene oxide)/poly(propylene oxide) (PEO/PPO) copolymers and its derivatives, and poly(ethylene glycol)/biodegradable polyester copolymers. PLGA-PEG-PLGA (PPP) triblock copolymer is the most widely studied thermosensitive hydrogel owing to its non-toxic, biocompatible, biodegradable, and thermosensitive properties.

### Biodegradable Polymers

Utilization of polymers as biomaterials has greatly impacted the advancement of modern medicine. Specifically, polymeric biomaterials that are biodegradable provide the significant advantage of being able to be broken down and removed after they have served their function (Ulery et al. 2011 J Polym Sci B Polym Phys 49: 832-864). Natural biodegradable polymers include fibrin, collagen, chitosan, gelatin and hyaluronan. Examples of synthetic polymers include Poly(lactic acid) (PLA), poly(glycolic acid) (PGA) (PGA), Poly(lactide-co-glycolide) (PLGA), Poly (Î-caprolactone) PCL, polyorthoesters, poly(dioxanone), poly(anhydrides), poly(trimethylene carbonate), polyphosphazenes, polyhydroxy butyrate (PHB), and poly-hydroxybutyrate-co-b-hydroxy valerate (PHBV).

### Blended Electrospun Microfibers

The blended electrospun microfibers are made by blending the thermosensitive hydrogel with the biodegradable polymer prior to electrospinning. An electric field is used to create a charged jet of polymer solution. As this jet travels in air, the solvent evaporates leaving behind a charged fiber that can be electrically deflected or collected on a screen. Fibers with a variety of cross sectional shapes and sizes are produced from different polymers. The diameter of these fibers may be in the range of 1-50 microns. For example, the fibers may have a diameter of about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm or the diameter is within a range bounded by any two of the preceding numerical values.

A ratio of thermosensitive hydrogel: biodegradable polymer in the blended microfibers can be 10:90, 15:85, 20:80, 25:75, 30:70, 35:65, 40:60, 45:55, 50:50, 55:45, 60:40, 65:35, 70:30, 75:25, 80:20, 85:15 or 90:10. In preferred embodiments, the ratio of thermosensitive hydrogel: biodegradable polymer is 65:35. In a particularly preferred embodiment, the electrospun fiber contains 65% PEG-PNIPAAm and 35% PCL.

A scaffold containing the hybrid fibers can have a thickness of about 0.5 mm, 1.0 mm, 1.5 mm, 2.0 mm, 2.5 mm, 3.0 mm, 3.5 mm, 4.0 mm, 4.5 mm, 5.0 mm, or 5.5 mm, 6.0 mm, 6.5 mm, 7.0 mm, 7.5 mm, 8.0 mm, 8.5 mm, 90 mm, 9.5 mm or 10.0 mm, or a thickness within a range bounded by any two of the preceding numerical values.

A scaffold containing the hybrid fibers can have a compressive modulus of between 40-80 kPa, for example, 40 kPa, 45 kPa, 50 kPa, 55 kPa, 60 kPa, 65 kPa, 70 kPa, 75 kPa and 80 kPa, or a compressive modulus within a range bounded by any two of the preceding numerical values.

### Advantages of Blended Electrospun Microfibers

As disclosed herein, thermosensitive PEG-PNIPAAm is incorporated with PCL prior to electrospinning and subsequent electrospinning of the mixture produces thick (˜2.5 mm) hybrid scaffolds with a micro-sized fibrous structure. The process does not require a step of UV-induced graft polymerization as in Yu et al. (2016 Materials Letters 164: 505-508) The result is blended PCL:PEG-PNIPAAm electrospun microfibers that have superior mechanical properties in cell/scaffold constructs as compared to scaffolds of only the pure forms of its constitutive components. We have demonstrated the utility of the hybrid scaffold by showing enhanced chondrogenic differentiation of human mesenchymal stem cells (hMSCs) cultured in the scaffolds. A particular ratio of PEG-PNIPAAm: PCL (i.e., containing 65% PEG-PNIPAAm) forms a hydrogel that completely fills the pores of the scaffolds and which has a compressive modulus of almost 3-fold greater than that of pure hydrogel. There was no basis provided by Yu et al. (supra) to mix PEG-PNIPAAm with PCL prior to electrospinning, let alone that the resulting blended electrospun microfibers would demonstrate the above advantageous features.

### Engineered Tissues

Engineered tissues can replace any hard (e.g., cartilage/bone) or soft tissue (e.g., skin/veins/arteries/organs) that may be damaged by disease or injury. Mesenchymal stem cells (MSCs) derive from embryonic stem cells and differentiate into cell lineages that form all connective tissues such as adipose tissue, cartilage, bone, skeletal muscles, and interstitial fibrous tissue. MSCs can be isolated from bone marrow, adipose tissue, deciduous teeth, and skeletal muscle. In addition to their multipotent nature, MSCs can self-renew and replicate for many passages without losing their stemness (pluripotency). MSCs were first isolated and identified as fibroblast-like cells that are adherent to cell culture Petri dishes. In humans, MSCs are typically aspirated from the bone marrow of the superior iliac crest, whereas murine MSCs are commonly aspirated from the mid-diaphysis of the tibia and femur. Upon exposure to various established induction media such as adipogenic, chondrogenic, osteogenic, and myogenic media, MSCs differentiate into distinct pathways and begin to express genes and protein markers characteristic of corresponding cell lineages.

Cartilage tissue may be grown by chondrogenic differentiation of mesenchymal stem cells (hMSCs). While bone marrow (BM) represents a well-documented source of cells for the regeneration and repair of skeletal tissues, a wide variety of alternatives, including adipose tissue (AT) (P. A. Zuk, M. Zhu, H. Mizuno et al., “Multilineage cells from human adipose tissue: implications for cell-based therapies,” Tissue Engineering, vol. 7, no. 2, pp. 211-228, 2001; P. A. Zuk, M. Zhu, P. Ashjian et al., “Human adipose tissue is a source of multipotent stem cells,” Molecular Biology of the Cell, vol. 13, no. 12, pp. 4279-4295, 2002), muscle (C. H. Evans, “Native, living tissues as cell seeded scaffolds,” Annals of Biomedical Engineering, vol. 43, no. 3, pp. 787-795, 2015), umbilical cord blood (S. Kern, H. Eichler, J. Stoeve, H. Klüter, and K. Bieback, “Comparative analysis of mesenchymal stem cells from bone marrow, umbilical cord blood, or adipose tissue,” STEM CELLS, vol. 24, no. 5, pp. 1294-1301, 2006; A. Erices, P. Conget, and J. J. Minguell, “Mesenchymal progenitor cells in human umbilical cord blood,” British Journal of Haematology, vol. 109, no. 1, pp. 235-242, 2000), umbilical cord Wharton's jelly (K. E. Mitchell, M. L. Weiss, B. M. Mitchell et al., “Matrix cells from Wharton's jelly form neurons and glia,” STEM CELLS, vol. 21, no. 1, pp. 50-60, 2003), dental pulp (S. Gronthos, M. Mankani, J. Brahim, P. G. Robey, and S. Shi, “Postnatal human dental pulp stem cells (DPSCs) in vitro and in vivo,” Proceedings of the National Academy of Sciences of the United States of America, vol. 97, no. 25, pp. 13625-13630, 2000), and periosteal tissue (C. L. Radtke, R. Nino-Fong, B. P. Esparza Gonzalez, H. Stryhn, and L. A. McDuffee, “Characterization and osteogenic potential of equine muscle tissue- and periosteal tissue-derived mesenchymal stem cells in comparison with bone marrow- and adipose tissue-derived mesenchymal stem cells,” American Journal of Veterinary Research, vol. 74, no. 5, pp. 790-800, 2013), have been explored for bone regeneration.

### EXAMPLES

### Thermosensitive Hydrogel Synthesis

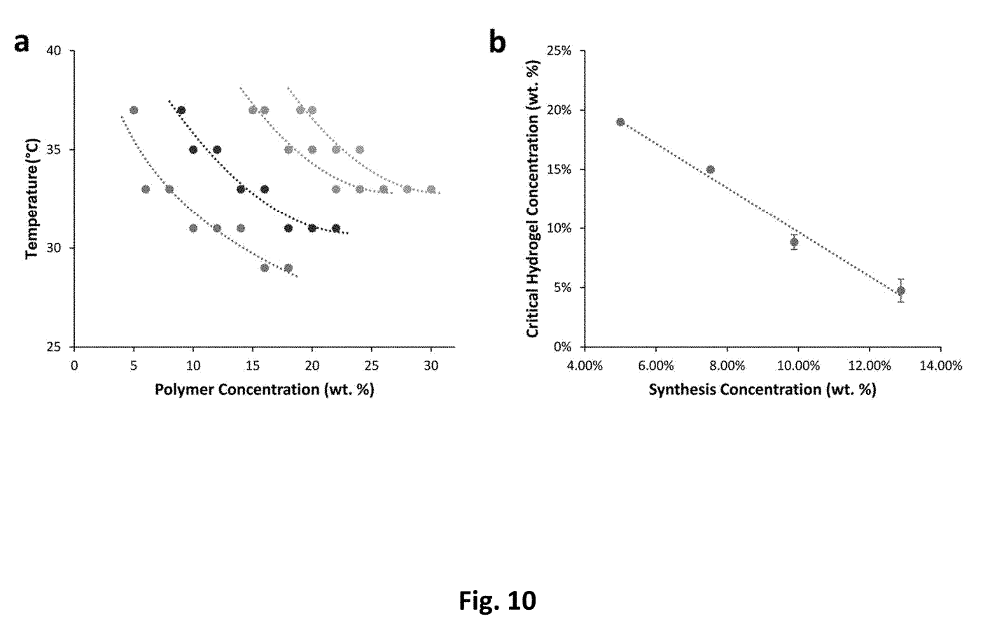

N-isopropylacrylamide (NIPAAm) (Sigma Aldrich) monomer and PEG-dimethacrylate (PEG-DMA, M.W.=8000) (Alfa Aesar) were utilized to synthesize a thermosensitive hydrogel polymer, PEG-PNIPAAm, using a modified free-radical polymerization protocol [22]. Briefly, NIPAAm and PEG-DMA were dissolved together in methanol under nitrogen atmosphere at various total precursor concentrations with a fixed NIPAAm:PEG-DMA ratios of 9:1 to optimize the gelation behaviors of the synthesized PEG-PNIPAAm (FIG. 10). Azobisisobutyronitrile (AIBN) (Sigma Aldrich) was added to the solution as a free-radical initiator. The reaction flask was heated to 65° C., and refluxed for 48 hours, followed by the evaporation of methanol. The resulting solid polymer was washed in n-hexane (Sigma Aldrich) at 45° C. for 12 hours to remove unreacted monomers. It was then grinded into a powder and vacuum-filtered with three rinses of n-hexane.

### Hydrogel Characterization

Fourier transform infrared (FTIR) spectroscopy and proton nuclear magnetic resonance (H NMR) spectroscopy were used to analyze the chemical structure of the synthesized PEG-PNIPAAm. Dry powder samples were used for FTIR analysis with a Nicolet 6700 FTIR Spectrometer (Thermo Fisher Scientific) to measure infrared absorbance peaks of the PEG-PNIPAAm. Alternatively, the polymer was dissolved in deuterated chloroform (Sigma Aldrich) at 20 wt. % and subjected to H NMR spectroscopy using a Varian Inova 400 NMR machine (Varian, Inc.). The H NMR data was analyzed using Mnova Analytical Chemistry Software (Mestrelab Research). Pure PEG-DMA and NIPAAm monomers were used as controls.

The lower critical solution temperature (LCST) of the thermosensitive PEG-PNIPAAm hydrogel was characterized at various solution concentrations (4-18 wt. % dissolved in Dulbecco's Modified Eagle Medium: nutrient mixture F-12 (DMEM/F12) (Fisher Scientific) at 4° C.) using a standard inversion assay under various temperatures. The temperature of the polymer solution was gradually increased from room temperature (23° C.) to 41° C., during which the vials containing the dissolved hydrogel solution were tilted 45° to determine gelation after every 2° C. temperature increase.

### Cell Viability In Hydrogel

Bone marrow-derived human MSCs (Sciencell) were used to determine the biocompatibility of the PEG-PNIPAAm hydrogel. PEG-PNIPAAm powder was sterilized by an exposure to gamma irradiation at a dose of 10 kGy [23] before being dissolved in sterile DMEM/F12 overnight at 4° C. The cells were inoculated into the polymer solution at a cell density of 12×10⁶ cells/mL with the final polymer solution concentrations of 5, 7.5 and 10 wt. %. Approximately 380 μl of the cell/hydrogel solution was plated in each well of a 24-well plate to form approximately 2 mm thick cell-laden hydrogel and incubated at 37° C. for 20 minutes to achieve gelation. An additional 500 μl of warm DMEM/F12 solution was then added to the cell/hydrogel specimens. The samples were cultured for 24 hours before being subjected to a live/dead cell assay using 2 μM Calcein-AM (Fisher Scientific) and 4 μM ethidium homodimer-1 (Fisher Scientific) in sterile phosphate buffer solution (PBS) (Fisher Scientific). The samples were incubated in the staining solution for 5 minutes at 37° C. Unbound excess stain solution was aspirated and washed with warm PBS. 10% formaldehyde in PBS solution was used to fix the cells for 30 minutes while maintaining the gelled structure of the hydrogel at 37° C. The fluorescently labeled samples were immediately imaged using a fluorescence microscope. Five different locations (center and four corners of a square) in three different focal planes from three biologically independent samples were utilized to quantify cell viability.

### Hybrid Scaffold Fabrication

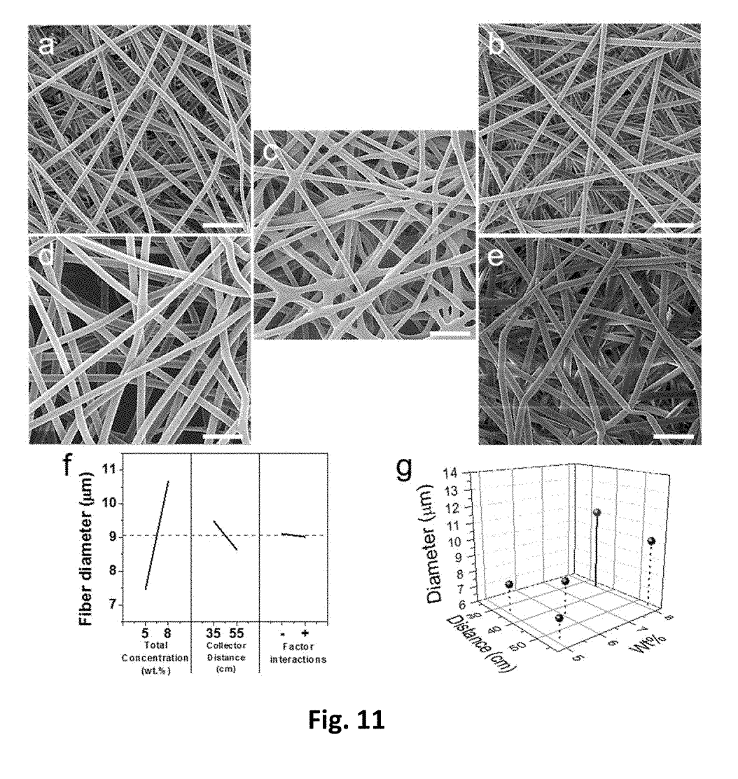

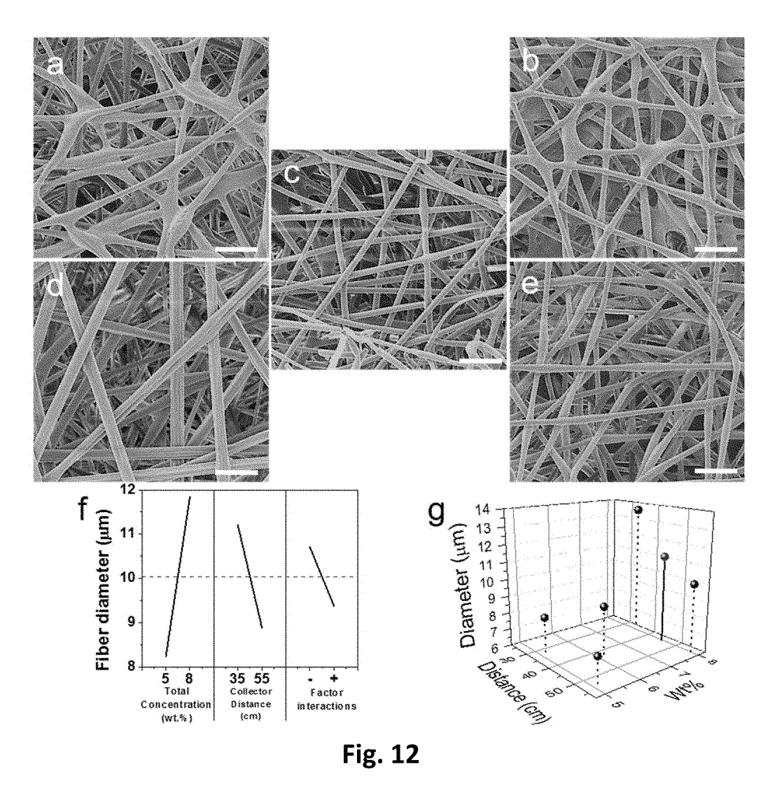

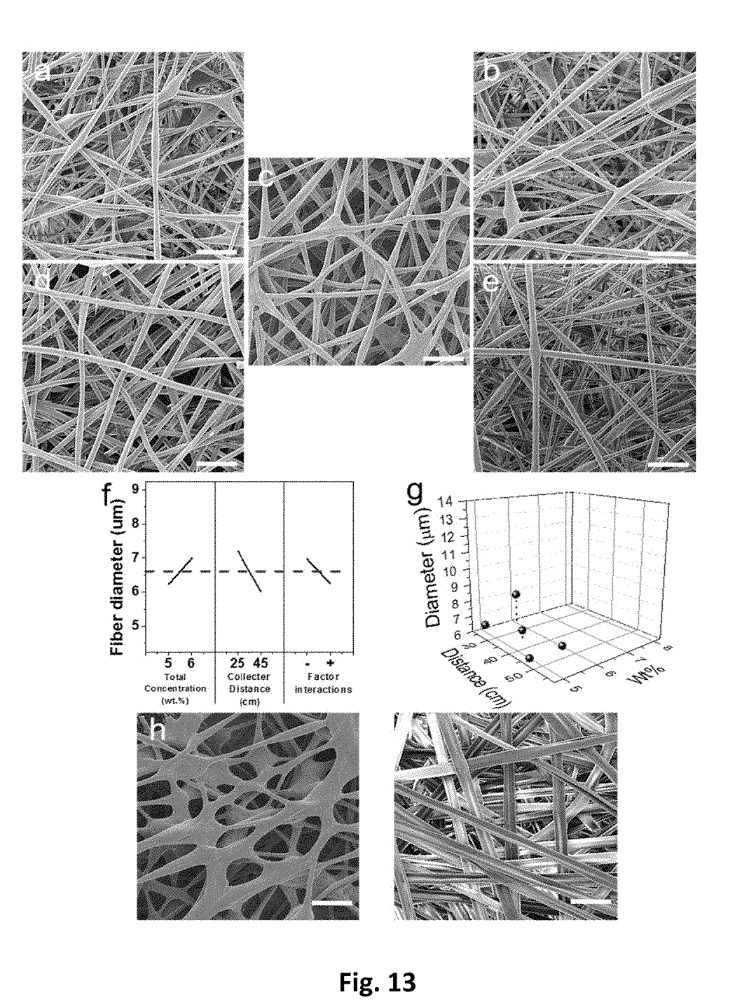

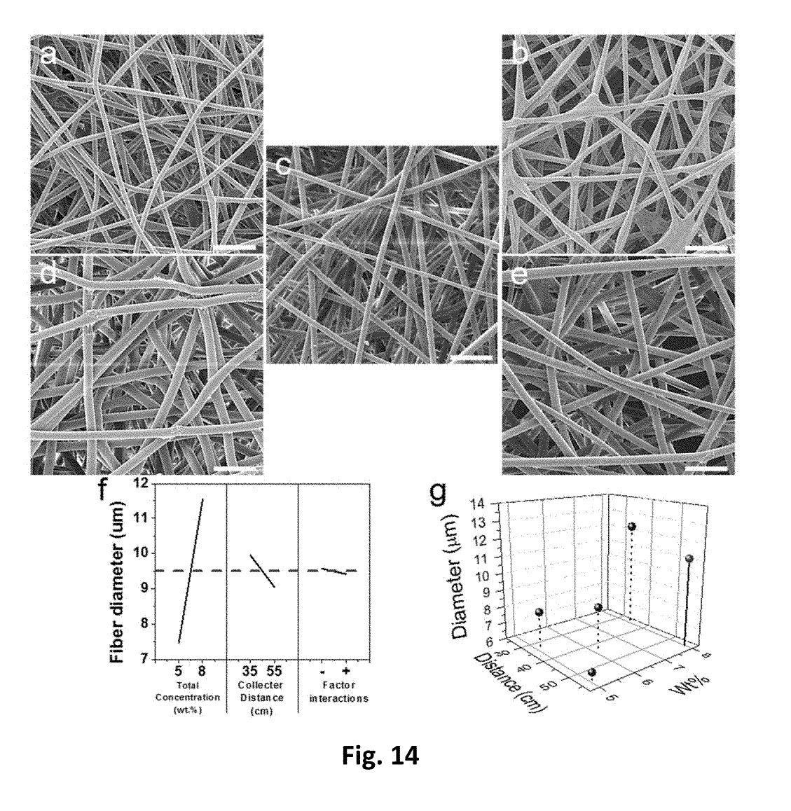

Different blending ratios of polymer solutions including but not limited to 25%, 50% and 65% PEG-PNIPAAm to PCL, were dissolved in chloroform at various overall polymer concentrations. Optimal electro spinning parameters, including overall polymer concentration (5-8 wt. %), solution feed rate (8-10 mL/hr), applied voltages (−9.5 to −13.0 kV), and spinneret-to-collector distance (25-55 cm), were experimentally determined by a design-of-experiment (DOE) approach for each blending ratio to achieve an appropriate average fiber diameter of 11 μm for uniform cell distribution upon cell seeding (Table 2 and FIGS. 11-14) [24-26]. Pure PCL scaffolds with similar morphology and fiber diameters were also electrospun as described previously and used as controls [26]. The electrospinning duration was adjusted to achieve a scaffold thickness of approximately 2.5 mm. The resulting fibrous mesh was then vacuum dried overnight at <30 mmHg to remove residual chloroform [27]. Individual cylindrical scaffold samples were then cut from the mesh using a 6 mm medical biopsy punch (Miltex).

### Hybrid Scaffold Characterization

Scaffold morphology and fiber diameters were evaluated using scanning electron microscopy (SEM) and ImageJ Software. Individual scaffold dimensions were measured with digital calipers and weighed before further testing. Scaffold porosity was determined using a liquid displacement method similar to our previous work [26]. Gelation of the hybrid scaffolds was induced by injecting 65 μL of DMEM/F12 solution onto each scaffold and incubating them at 37° C. for 20 minutes. To visualize the hydrogelation within the hybrid scaffolds, the constructs were immediately snap frozen in liquid nitrogen and lyophilized for 2 days using a Freezone 1 lyophilizer (Labconco) before being imaged by SEM. Alternatively, to visualize the PCL structure of the hybrid scaffolds, the hydrogel component was removed with a cold PBS wash overnight on a shaker plate, followed by SEM imaging.

### Mechanical Characterization

Mechanical characterization of the scaffolds was performed as previously described [26]. Briefly, a custom-made compression system composed of a Soloist Motion servo controller and a Z-axis lift stage (Aerotech), was utilized to subject the scaffold to a sinusoidal compressive strain of 0.05 at 1 Hz while the force was recorded using a load cell (Honeywell). The characterization of dry scaffolds was performed at room temperature, whereas the characterization of hydrated scaffolds was performed after each scaffold was injected with 65 μL of warm DMEM/F12 media and incubated for 20 minutes at 37° C. The compression setup was placed inside a cell culture incubator to maintain the temperature at 37° C. Alternatively, the hydrated scaffolds were cooled down to room temperature to examine the effect of the thermosensitive hydrogel on the overall mechanical properties of the hybrid scaffolds. Furthermore, the hybrid and pure PCL scaffolds after two weeks of cell culture were similarly subjected to mechanical characterization.

Chondrogenesis of hMSCs in the Hybrid Scaffolds

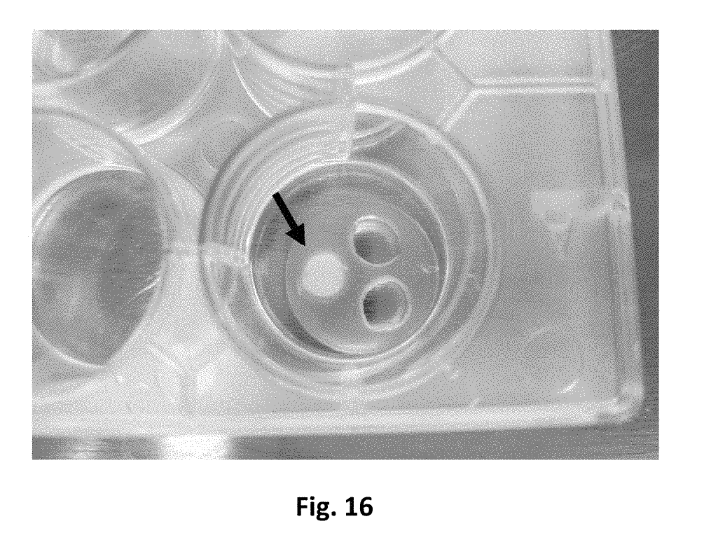

Scaffolds were sterilized by gamma irradiation at 10 kGy [23]. Approximately 12×10⁶ hMSCs were suspended in 1 mL of DMEM/F12 media and 65 μL of the cell solution was seeded into each scaffold (FIG. 1). The cell-seeded scaffolds were immediately incubated at 37° C. for 20 minutes before an additional 600 μL of media was supplemented into each well. During this incubation period, no spillage of cell-containing solutions from the scaffolds was observed, likely due to capillary forces generated by the macropores within the scaffolds. Samples of cell/scaffold constructs were snap frozen in liquid nitrogen and cryosectioned to determine the cellular distribution throughout the thickness of the scaffolds upon seeding, as previously described [26]. Alternatively, the cell/scaffold constructs were pre-cultured in DMEM/F12 media for three days before subjecting the cells to chondrogenic media (low glucose DMEM media with 0.1 μM Dexamethasone (Sigma Aldrich), 200 μM ascorbic acid (Sigma Aldrich), 10% fetal bovine serum (Fisher Scientific), 1% penicillin/streptomycin (Fisher Scientific) and 10 ng/mL TGF-β (Peprotech Inc.)) for 2 weeks. PDMS molds having cylindrical holes with a diameter of 6 mm were utilized to cast cell-laden pure PEG-PNIPAam hydrogel at approximately 6.5 wt. %, with a thickness of 2 mm (FIG. 16). After the culture period, the cell/scaffold constructs were subjected to biomechanical analysis as described earlier, or biochemical and histological analyses as described below.

### Gene Expression Analysis

After 14 days of culture, mRNA was extracted from the samples using the Qiagen RNeasy MiniKit according to the manufacturer's protocol. The cDNA was synthesized by using the iScript cDNA Synthesis Kit (Bio Rad). The synthesized cDNA was subjected to real time-polymerase chain reaction (rt-PCR) using custom SYBR Green primers to determine chondrogenic gene expression. The primers used were collagen type II (COL2A1; forward: CAACACTGCCAACGTCCAGAT, SEQ ID NO: 1 and reverse: CTGCTTCGTCCAGATAGGCAA, SEQ ID NO: 2), aggrecan (ALAN; forward: TGAAACCACCTCTGCATTCCA, SEQ ID NO: 3 and reverse: GACGCCTCGCCTTCTTGAAAT, SEQ ID NO: 4), and SOX9 (forward: TGCTCAAAGGCTACGACTGGA, SEQ ID NO: 5 and reverse: TTGACGTGCGGCTTGTTCT, SEQ ID NO: 6). GAPDH (forward: ATGGGGAAGGTGAAGGTCG, SEQ ID NO: 7 and reverse: TAAAAGCAGCCCTGGTGACC, SEQ ID NO: 8) was used as an endogenous control gene. The data were subjected to gene expression analysis by the comparative threshold method [28].

### Histological Analysis

The cell/scaffold constructs were washed twice with warm PBS and then fixed in 10% buffered formalin at 37° C. for 30 minutes. The samples were snap frozen in liquid nitrogen, embedded in OCT compound, and cryosectioned. The sectioned samples were quickly heated up to 37° C. to minimize the dissolution of hydrogel, and subjected to either Alcian Blue or Safranin-O staining as previously described [25, 26]. The stained samples were then imaged using a microscope in brightfield mode.

### Statistical Analysis

All experiments were performed with at least three biologically independent samples, and represented as an average ±standard deviation (SD), except for gene expression in which the standard error of mean (SEM) for n=6 (biologically independent triplicate with technical duplicate) was used. For mechanical testing, n=6 was used. The data were subjected to ANOVA with Tukey's post-hoc test using the SPSS software (IBM) to determine statistical significance (p<0.05).

### Results

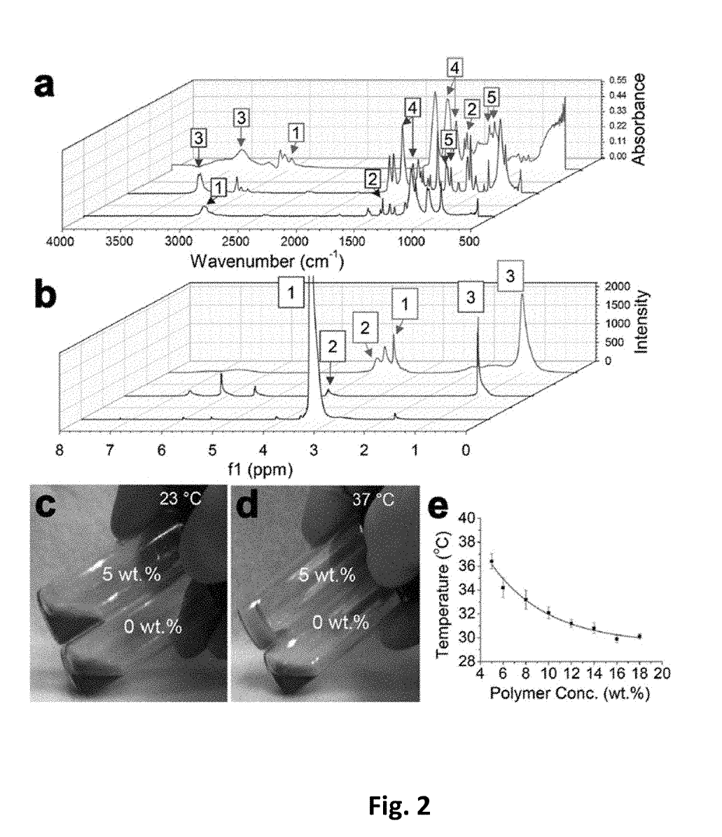

The aim of this study was to develop a hybrid scaffold that exploits the superior bioinductiveness of hydrogels and the mechanical stability of electrospun microfibers (FIG. 1). To minimize the number of steps required in traditional hydrogel processing techniques such as chemical or UV-irradiation crosslinking, a thermosensitive hydrogel, PEG-PNIPAAm, was utilized. First, the molecular structure of the synthesized PEG-PNIPAAm polymer was analyzed by FTIR and H NMR spectroscopy. The characteristic peaks of PEG-PNIPAAm in its FTIR spectrum were compared to those of the precursor components, NIPAAm and PEG-DMA (FIG. 2a). The major functional molecular groups observed in PEG-PNIPAAm were derived from PEG-DMA due to the stretching of the epoxy group at 2948 cm⁻¹ (FIG. 2a, labeled 1, C—H stretching) and 1466 cm⁻¹ (labeled 2, C—H stretching) [29, 30]. In addition, the peaks associated with NIPAAm's broad N—H stretching vibration peak around 3435 cm⁻¹ (labeled 3), amide groups at 1650 cm⁻¹ (Amide I,C═O stretching) and 1550 cm⁻¹ (labeled 4, Amide II, N—H bending), and isopropyl group splitting at 1387 cm⁻¹ and 1367 cm⁻¹ (labeled 5) were also observed in the synthesized PEG-PNIPAAm [31-33]. A similar validation of the molecular structure was performed with an H NMR analysis (FIG. 2b). The most prominent peaks, PEG-DMA's peak associated with —H of the PEG main chain located at 3.6 ppm (labeled as 1 in FIG. 2b) [22, 34], as well as, NIPAAm's C—H proton at 3.8 ppm and methylene proton peak at 1.1 ppm (labeled as 2 and 3, respectively) [22, 35], were all present in PEG-PNIPAAm. The intensity ratio of the peaks for 1-ethylene protons associated with NIPAAm and 1-ethylene protons associated with PEG-DMA were used to determine an approximate ratio of 650:1 between PNIPAAm and PEG crosslinking chains in the synthesized polymer.

The hydrogelation behavior of PEG-PNIPAAm was characterized by dissolving PEG-PNIPAAm in cell culture media (DMEM/F12) at various concentrations and subjecting them to an inversion assay at various temperatures (FIG. 2c, d, e). The LCST of each solution dissolved at 4° C. (FIG. 2c) was determined by incubation at increasing temperatures for five minutes until gelation was observed (FIG. 2d). The solution of pure DMEM/F12 (0 wt. %) was used as a control. The temperatures at which the thermosensitive hydrogel solidified were plotted with respect to solution concentration to determine the LCST (FIG. 2e). The highest concentration tested (18 wt. %) formed a gel at 29° C. As the concentration decreased, the temperature required to induce gelation gradually increased. A solution of 5 wt. % was the minimum concentration required to form a hydrogel at 37° C.

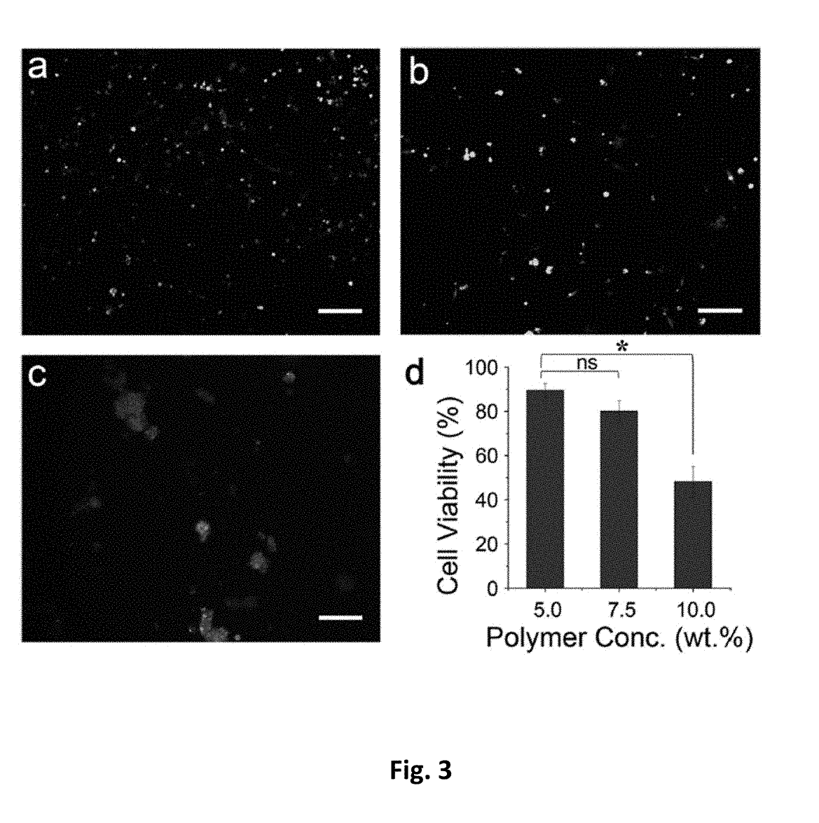

Based on the determined LCST of PEG-PNIPAAm, cell viability was examined with hMSCs cultured in various hydrogel concentrations (5, 7.5 and 10 wt. %) using a Live/Dead cell assay (FIG. 3). Interestingly, greater cell clustering within the 3D constructs was observed as the hydrogel concentration increased. The quantification of live/dead cells by image analysis demonstrated that the hydrogel at 5 wt. % supported the highest cell viability at approximately 90%. Cells cultured in 7.5 wt. % hydrogel constructs were approximately 80% viable after 24 hours of culture, but was statistically insignificant from the 5 wt. % condition. In contrast, the cells cultured in 10 wt. % hydrogel constructs exhibited a significant decrease in cell viability at approximately 50%.

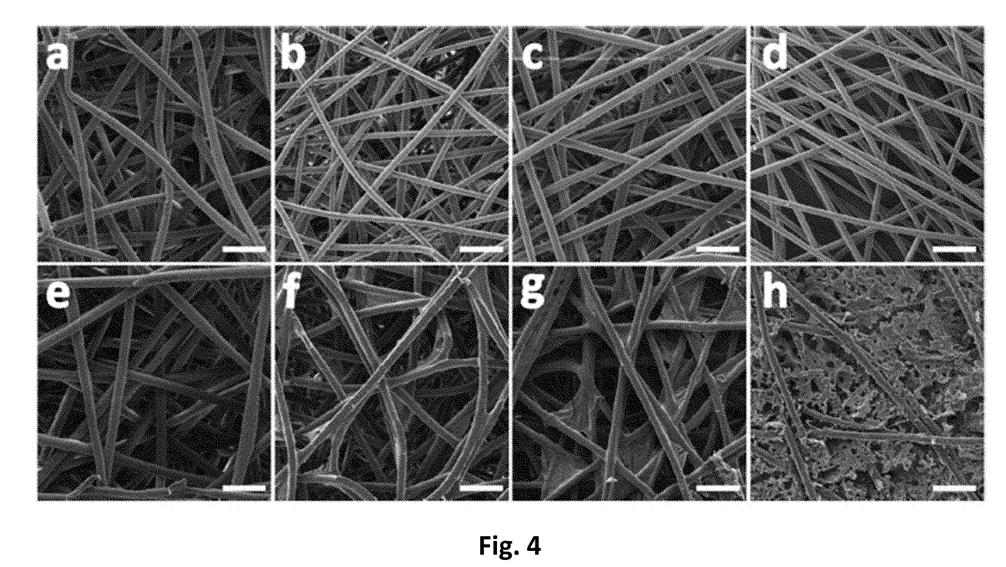

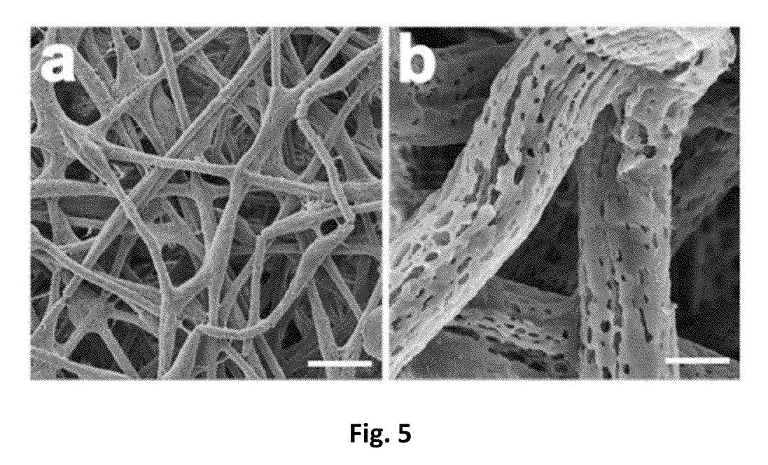

After the chemical and biological characterization, the PEG-PNIPAAm hydrogel was composited into electrospun PCL to form hybrid scaffolds for 3D cell culture. Solutions with different blended mass ratios of PEG-PNIPAAm:PCL were synthesized to vary the scaffold characteristics (Table 1, FIG. 4). The optimization of electrospinning parameters was performed by a design-of-experiment (DOE) approach for each PEG-PNIPAAm:PCL ratio (Table 2, FIGS. 11-14). Solution properties, such as polymer concentration and solvent system, and electrospinning parameters, such as solution feeding rate, applied voltages and collection distance, were optimized to synthesize fiber diameters of approximately 11 μm, with a scaffold porosity of approximately 90%, to promote complete cellular infiltration upon seeding without impacting the scaffolds' mechanical properties as previously shown [26]. Solutions with a PEG-PNIPAAm concentration above 70% failed to produce fiber diameters greater than 8 μm, and were excluded from subsequent analyses. The individual electrospun fibers from all conditions exhibited a typical cylindrical morphology (FIG. 4a, b, c, d). Various properties of the hybrid scaffolds having three representative PEG-PNIPAAm:PCL ratios including 25%, 50% and 65% (represented by PEG-PNIPAAm percentages) were examined as compared to pure PCL control scaffolds (0%) (Table 1). Based on the porosity of each scaffold condition, the appropriate volume of cell culture media was added to fill the pores, followed by incubation at 37° C. for 20 minutes. The media was maintained within the scaffold during incubation, likely due to capillary force generated by the porous fibrous structure of the scaffold. The samples were subsequently lyophilized to reveal the structural changes of the scaffolds due to the hydrogelation of PEG-PNIPAAm (FIG. 4e, f, g, h). As expected, the hydration of pure PCL control did not change the scaffold structure. In contrast, the hydration of the hybrid scaffolds resulted in the dissolution and gelation of PEG-PNIPAAm as evident from the structural change, i.e., formation of nanostructured hydrogel within the micropores of the scaffolds. The pores of 65% hybrid scaffold were completely filled by the hydrogel after hydration as depicted in the SEM image (FIG. 4h). The structural integrity of the 65% hybrid scaffold after hydrogel formation was determined by subsequently removing the hydrogel component PEG-PNIPAAm from the scaffold by cold PBS washes (FIG. 5). Although there were pit formations on the surface of the electrospun fibers after washing, the overall structure of the scaffold was maintained. The 65% PEG-PNIPAAm scaffold had minimal dimensional changes upon hydration with an approximately 10% reduction in height from its original dimensions. The PEG-PNIPAAm concentration within the 65% hybrid scaffold upon hydration, calculated from the blending ratio and porosity of the scaffold, was approximately 6.2 wt. %, above the critical gelation concentration of 5 wt. %.

TABLE 1
Morphological characterization of PEG-PNIPAAm/PCL scaffolds
with various blending ratios.

| Ratio of PEG- | Avg. Fiber | Avg. Scaffold |
|---|---|---|
| PNIPAAm | Dimeter (μm) | Porosity (%) |
| 0% | 11.6 ± 1.0 | 92.7 ± 0.2 |
| 25% | 11.1 ± 0.5 | 94.9 ± 4.9 |
| 50% | 11.2 ± 0.6 | 92.8 ± 3.8 |
| 65% | 11.2 ± 0.8 | 89.7 ± 5.2 |

TABLE 2
Design of experiment (DOE) for electrospun fiber diameter optimization
of different PEG-PNIPAAm:PCL ratios. Overall polymer concentration
and collector distance were used as variables. Values shown are average
fiber diameter in μm.

| Distance (cm) | | |
|---|---|---|
| 35 | 45 | 55 |

25% PEG-PNIPAAm Blended Fibers

| | | | | |
|---|---|---|---|---|
| Poly. Conc. (wt. %) | 5 | 7.8 ± 1.0 | | 7.4 ± 0.8 |
| | 6 | | 8.2 ± 1.2 | |
| | 8 | 11.1 ± 0.5 | | 10.2 ± 0.6 |

50% PEG-PNIPAAm Blended Fibers

| | | | | |
|---|---|---|---|---|
| Poly. Conc. (wt. %) | 5 | 8.4 ± 2.3 | | 7.7 ± 1.9 |
| | 6 | | 9.1 ± 0.8 | |
| | 8 | 13.6 ± 3.0 | 11.2 ± 0.6 | 10.0 ± 3.0 |

65% PEG-PNIPAAm Blended Fibers

| | | | | |
|---|---|---|---|---|
| Poly. Conc. (wt. %) | 5 | 8.3 ± 1.3 | | 6.6 ± 0.8 |
| | 6 | | 8.7 ± 0.6 | |
| | 8 | 12.3 ± 1.0 | | 11.1 ± 1. 0 |

75% PEG-PNIPAAm Blended Fibers

| | | Distance (cm) | | |
|---|---|---|---|---|
| | | 25 | 35 | 45 |
| Poly. Conc. (wt. %) | 5 | 6.5 ± 1.9 | | 6.0 ± 1.4 |
| | 5.5 | | 6.6 ± 0.8 | |
| | 6 | 7.9 ± 0.9 | | 6.0 ± 1.6 |

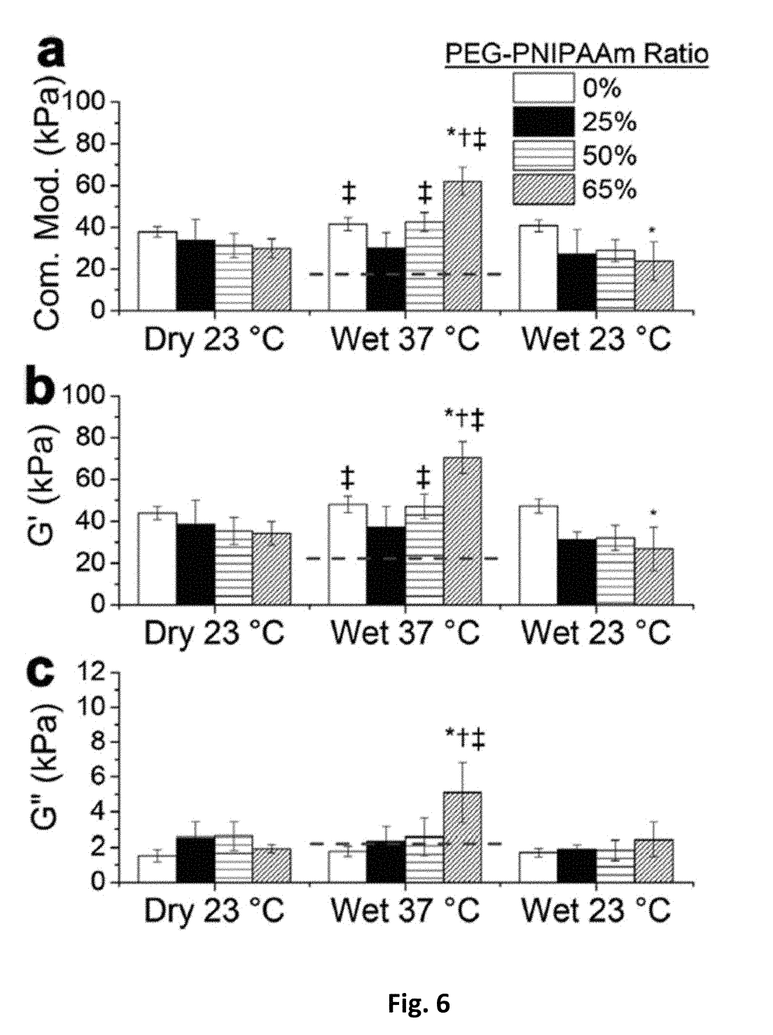

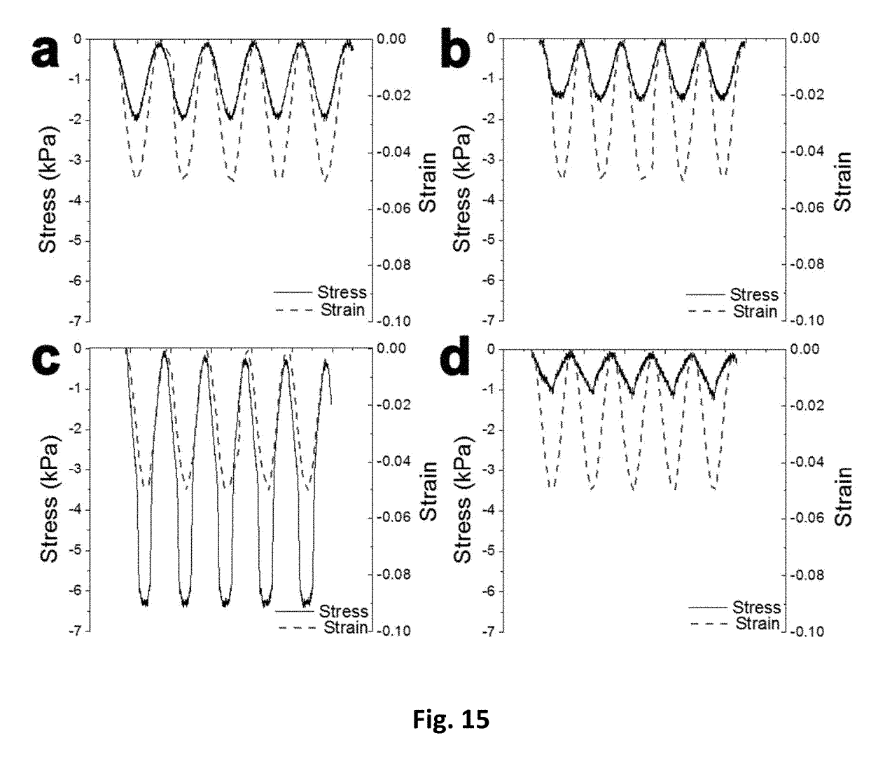

Mechanical testing of the hybrid scaffolds was performed to determine the effects of hydration of the hydrogel component on the overall mechanical properties of the scaffolds. The scaffolds were subjected to a dynamic compressive strain of 0.05 at 1 Hz to determine the compressive moduli, which were further decomposed to the elastic and viscoelastic moduli as previously shown (FIG. 6 and FIG. 15) [26]. As-synthesized samples (Dry 23° C.) of pure PCL, and 25%, 50% and 65% PEG-PNIPAAm hybrid scaffolds exhibited similar compressive moduli of approximately 35-40 kPa. However, hydration and incubation of the scaffolds at 37° C. (Wet 37° C.) resulted in a significant increase in the compressive modulus of the 65% hybrid scaffold due to the gelation of PEG-PNIPAAm. Specifically, the compressive modulus of the 65% hybrid scaffolds, 62.1±6.7 kPa, increased by close to 2-fold from its dry modulus of 29.8±4.6 kPa, was significantly higher than both that of pure PCL and other PEG-PNIPAAm scaffolds in the same condition. Meanwhile, the 25% scaffold remained at 30.1±7.4 kPa, relatively similar to its dry compressive modulus, likely due to the PEG-PNIPAAm concentration, once dissolved within the pore volume of the scaffold, being below the 5 wt. % minimum concentration for its phase transition to a gel state. The 50% hybrid scaffold exhibited a slight increase to 42.6±4.5 kPa upon hydration. In comparison, pure PEG-PNIPAAm hydrogel constructs exhibited a significantly lower compressive modulus of 19.3±4.2 kPa, as compared to the scaffolds containing electrospun PCL fibers. The compressive modulus of the 65% hybrid scaffolds significantly decreased to approximately 25 kPa, when the temperature was returned to 23° C. (Wet 23° C.), slightly below its as-synthesized dry compressive modulus. In contrast, the pure PCL scaffold maintained approximately 40 kPa at both 37 and 23° C. under the hydration conditions. The elastic (G′, FIG. 6b) and viscoelastic (G″, FIG. 6c) moduli were also derived from the mechanical data. As expected, the pure PCL samples retained their elastic and viscoelastic properties throughout all conditions. In contrast, the 65% hybrid scaffold exhibited a statistically significant increase in viscoelasticity upon hydration, indicating more efficient gelation within the scaffolds as demonstrated from the morphological observation (FIG. 4).

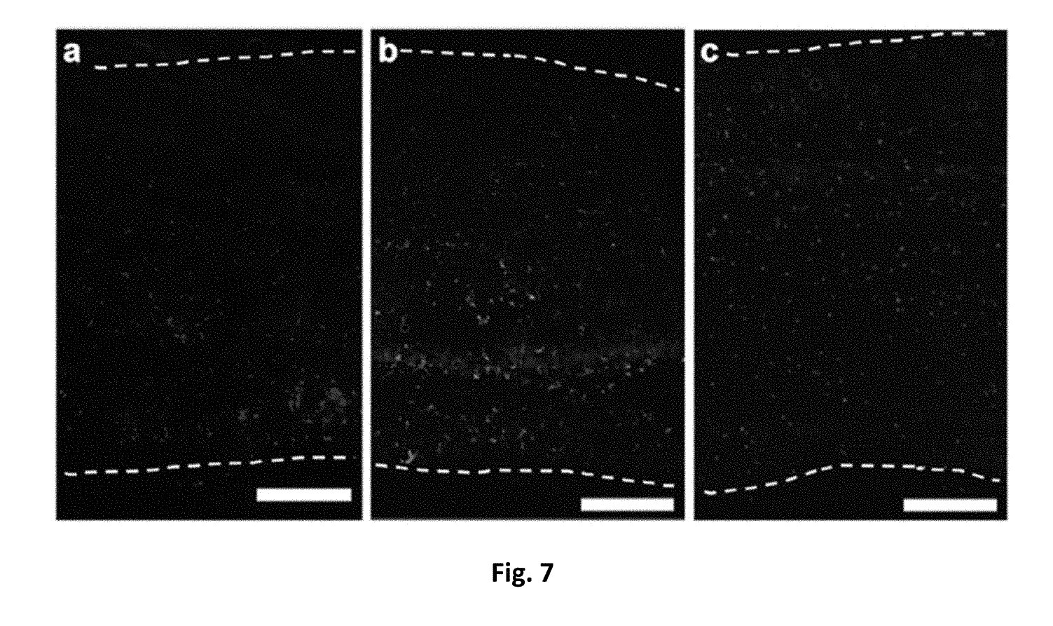

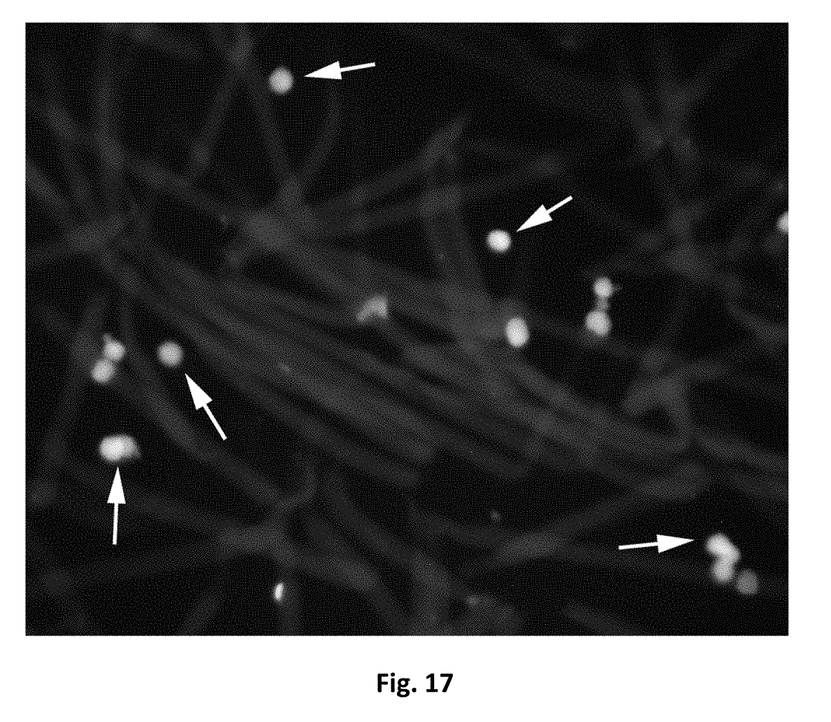

Another important characteristic of an optimal scaffold is its ability to allow uniform cellular infiltration within the structure [24]. To examine such uniform cell seeding, hMSCs were seeded in the hybrid scaffolds having various blend ratios. Cellular localization was determined by fluorescence imaging of the vertical cross-sections of the cell/scaffold constructs using DAPI nuclear staining (FIG. 7). Cells seeded in both the 25% and 50% hybrid scaffolds showed sedimentation of the seeded cells near the bottom of the scaffolds. In contrast, cells seeded in the 65% hybrid scaffold penetrated the entire thickness and uniformly distributed throughout the thickness of the scaffold. The seeded cells were localized both in the pores (likely filled by the hydrogel) and on the fibers (FIG. 17).

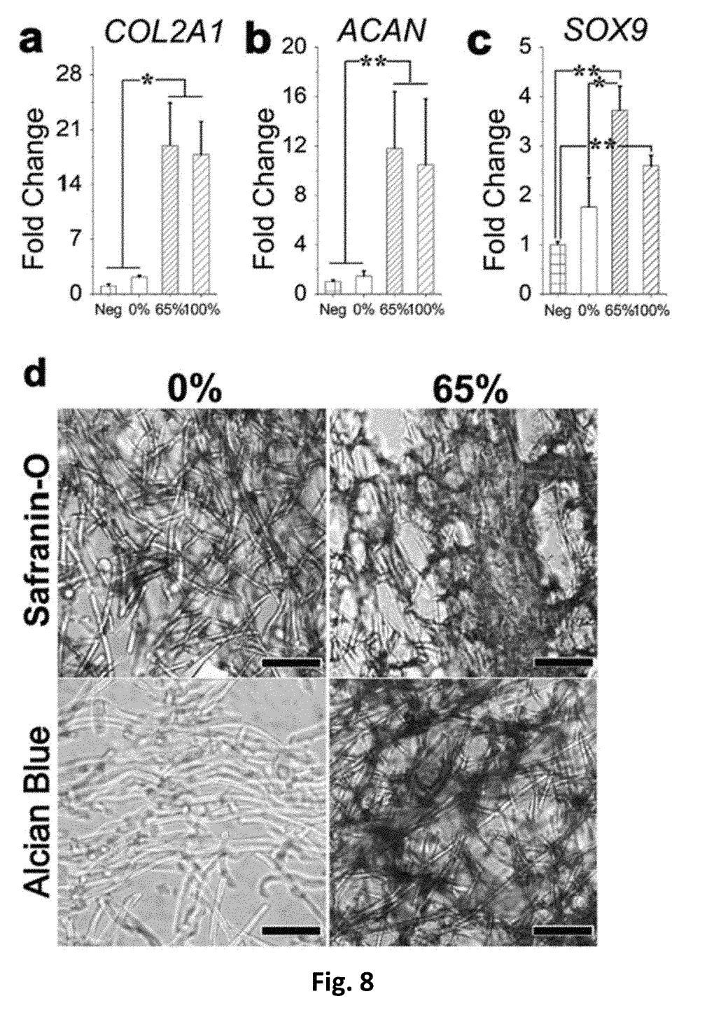

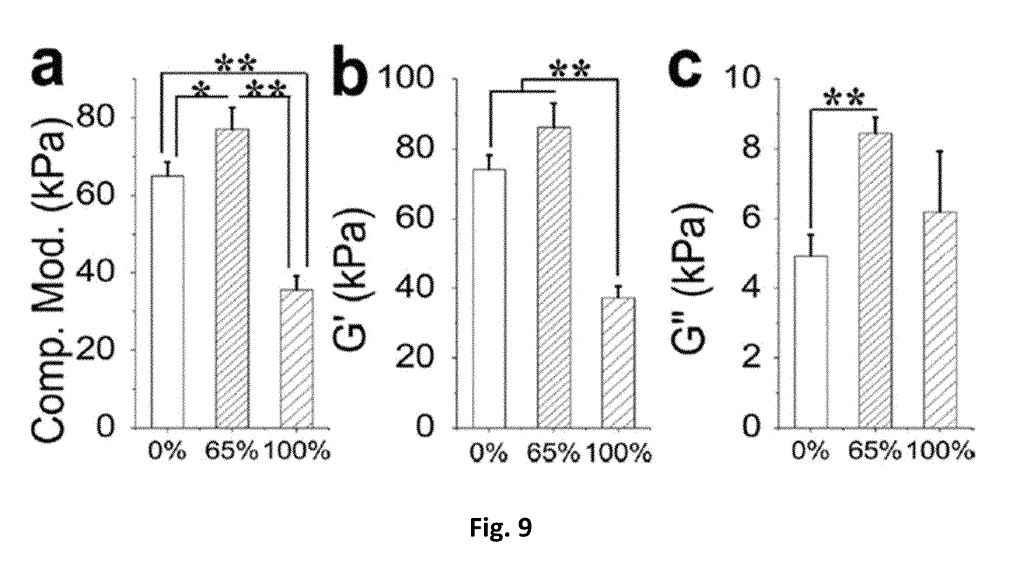

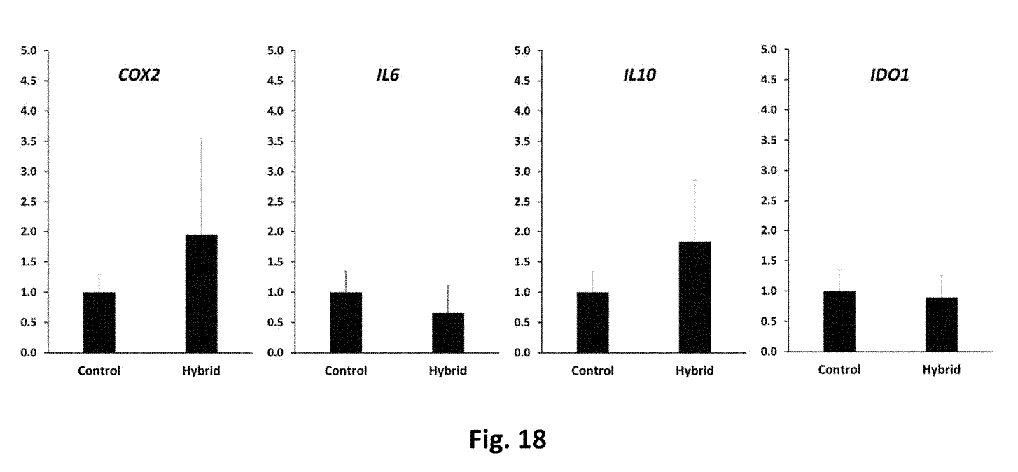

Based on the results from the scaffold characterizations, the 65% hybrid scaffolds were utilized to test their performance in tissue morphogenesis. Approximately 8×10⁵ hMSCs were seeded in either pure PCL (0%) or 65% hybrid scaffolds with a similar thickness of 2.5 mm, and the hMSC/scaffold constructs were subjected to chondrogenic conditions. The live-dead cell assay showed a cell viability of 89±3%, a value similar to 5 wt. % hydrogel as shown in FIG. 3. Furthermore, they hybrid scaffold did not induce acute inflammatory responses of hMSCs (FIG. 18). After a two week culture period, chondrogenic gene expression including collagen type II (COL2A1), aggrecan (ACAN) and SOX9 was significantly upregulated in the cells cultured in the 65% hybrid scaffolds to the similar levels exhibited by cells cultured in pure PEG-PNIPAam hydrogels (100%), as compared to those in pure PCL controls (0%) (FIG. 8a, b, c). At the protein level, the deposition of chondrogenic extracellular matrix (ECM) was enhanced in the 65% hybrid scaffolds as evident from greater intensities of Safranin-O and Alcian blue stains for glycosaminoglycan (FIG. 8d). As expected from the gene and protein expression results, the culture of hMSCs in the 65% hybrid scaffold resulted in greater compressive moduli and significantly greater viscoelasticity as compared to that of the cell-cultured pure PCL (FIG. 9). Although the cells cultured in the pure hydrogels expressed similar high levels of chondrogenic gene expressions as compared to the hybrid scaffolds, intrinsically inferior structural integrity resulted in the lowest compressive modulus among the conditions compared.

### Discussion

In conjunction with biochemical factors, the physical environment of the cells in engineered tissues, such as scaffold stiffness, pore size and its spatial arrangement, significantly affects tissue morphogenesis. The dimensionality of the scaffolds, which governs the availability of adhesion sites in 3D, has especially been shown to alter intracellular signaling cascades as compared to those in 2D [36-38]. In fact, stem cell behaviors including self-renewal, migration, and more importantly differentiation, are partly regulated by 3D distribution of adhesion sites [39]. Chondrogenesis of MSCs has been particularly shown to be enhanced by 3D encapsulation of the cells as compared to a monolayer culture, likely due to a similar cellular arrangement found in the native environment, where individual cells are surrounded by hydrated ECM. The 3D encapsulation of MSCs maintains a spherical cell structure which is believed to disrupt stress actin fiber formation within the cell, activating various signaling pathways including the p38 MAPK pathway, and inhibiting the ERK and Rho kinase pathways [40-43]. Such activation/inhibition of the signaling cascades collectively upregulates the activity of SOX9, the major chondrogenic transcription factor for the ECM associated genes: COL2A1 and ACAN. For this reason, various types of hydrogels have been utilized for cartilage tissue engineering applications [13, 15, 16]. Indeed, the hybrid scaffolds significantly enhanced the chondrogenesis of MSCs as evident from comparable expression of chondrogenic genes to that from the cells in the pure hydrogel, resulting in superior overall mechanical properties as compared to those of cell/pure PCL scaffold constructs. Although the modulus of the cell/hybrid scaffold constructs did not reach that of human articular cartilage after 2 weeks of culture, we expect to approach the native properties faster with a longer culture duration by using the hybrid scaffold as compared to either pure form of PCL or PEG-PNIPAAm.

Chemical and UV crosslinking strategies are popular choices for cell encapsulation in hydrogels. However, their dependency on diffusion kinetics of the chemical crosslinkers or the penetration depth of UV light, limits the size of cell/hydrogel constructs that can be manufactured having uniform cellular infiltration and material properties [44, 45]. In this regard, thermosensitive hydrogels offer an alternative strategy for uniform cellular encapsulation due to the relatively fast and thickness-independent thermal transfer in aqueous conditions. Among many thermosensitive hydrogels considered, PEG-PNIPAAm was employed in this study because of its biocompatibility, chemical modifiability, solvent miscibility for electrospinning, and its reverse thermo-sensitivity. PEG-PNIPAAm utilizes the thermosensitive gelation properties of PNIPAAm and the hydrophilicity of PEG for improved water retention. Both of the individual components, PEG and PNIPAAm, as well as their conjugated form, are biocompatible with a limited immune response from the body [22, 46-48]. Through a cell viability assay, we also demonstrated the minimal cytotoxicity of PEG-PNIPAAm except the noticeable decrease in cell viability at a high hydrogel concentration, which is likely due to hydrogel syneresis causing dehydration within the gel [49].

Our cell seeding results in the hybrid scaffold demonstrated the capability of PEG-PNIPAAm to uniformly encapsulate seeded cells in 3D and support their viability. The hybrid scaffold judiciously optimized for blending composition and fiber diameter, enabled uniform cellular infiltration throughout the thickness of the constructs upon cell seeding. Notably, the process does not require any additional steps for cell seeding, unlike typical hydrogel systems, where the use of a mold is necessary to maintain the scaffold shape. More importantly, encapsulation of cells within the hydrogel of the hybrid scaffold further enhanced the chondrogenic differentiation of hMSCs, as evident by the gene expression analysis and histology, demonstrating superior bioinductivity over pure PCL scaffolds.

In addition to its utility in mold-less cell encapsulation in 3D, the incorporation of PCL into PEG-PNIPAAm enhanced the mechanical properties of the hybrid scaffolds. One approach to address the relatively weak mechanical properties of hydrogels utilizes the supplementation of the hydrogel with supporting materials, including electrospun fibers [50]. Slow biodegradable polymers including PCL and its conjugates have been used in the form of either a mesh or cut segments to reinforce various hydrogels [51]. The inclusion of electrospun fibers, typically nanofibers, has been shown to increase the mechanical modulus of the hydrogel constructs, as compared to their pure forms. Recently, a few studies have electrospun a blend of a hydrogel precursor and a supporting polymer to synthesize a monolithic scaffold. Although this strategy enhanced the mechanical properties of the scaffolds upon hydration, the inherently small pore sizes of the nanofibrous structure typically prevented cellular infiltration, negatively impacting 3D tissue morphogenesis. In this regard, our previous studies have demonstrated the utility of electrospun microfibrous scaffolds for uniform cellular distribution, upon seeding via capillary action of the porous structure [24-26]. The limitation of producing microfibrous hydrogel structure by electrospinning is that most hydrogel precursors possess high conductivity in electrospinning solutions, typically resulting in small fiber diameters. In the present study, a careful screening of electrospinnable hydrogel polymers and solvent compatibility with supporting polymers, enabled the synthesis of the hybrid scaffolds composed of microfibers. In fact, the compressive modulus of the 65% hybrid scaffold composed of microfibers was almost 3-fold greater than that of pure hydrogel. Moduli, especially the viscoelastic modulus, were considerably enhanced as compared to that of the pure PCL scaffolds, likely due to the hydrogel filling the pores of the microfibrous PCL backbone.

Overall, the developed hybrid scaffold offers advantages over pure hydrogel or pure PCL electrospun fibrous scaffolds for enhanced processability and improved stem cell behaviors. Unlike other hydrogel systems, the hybrid scaffold contains a non-fragmented supporting structure of electrospun microfibers that provides mechanical stability. Furthermore, such structural integrity simplifies the processing steps for 3D cell encapsulation in the hydrogel, by removing the necessity of using molds during gelation to shape the construct. It also accommodates uniform cell seeding throughout the scaffold while providing a hydrogel network to mimic native ECM. The hybrid scaffold developed in this study, therefore, potentially offers a platform for tailored tissue scaffolding with easy modification of the hydrogel chemistry for functional enhancement, such as ligand incorporation for cell type-specific adhesion and crosslinking density for mechanical property modulation. At the same time, it mediates the inherent shortcomings of other hydrogel systems, i.e., mechanical instability and a multi-step fabrication process. Although MSC-derived chondrogenesis was investigated in this study to demonstrate the functionality of the developed hybrid scaffold, it is possible to expand the customization of either the hydrogel or the polymer backbone to optimize the scaffold for different applications and desired tissue types.

### Conclusions

In this study, we have developed a hybrid scaffold system that combines the superior bioinductivity of a hydrogel with the mechanical stability of electrospun fibers. The combination of thermosensitive PEG-PNIPAAm and PCL microfibers in a monolithic form enabled 3D cell encapsulation within the hydrogel by a simple cell seeding process. The hybrid scaffold improved the mechanical properties of cell/scaffold constructs while enhancing chondrogenic differentiation of hMSCs. Therefore, this novel scaffolding strategy may provide an opportunity to develop off-the-shelf, easy-to-use tissue scaffolding systems for various engineered tissues that benefit from 3D cell encapsulation within hydrogels.

### REFERENCES

[1] Jin K, Simpkins J W, Ji X, Leis M, Stambler I. The critical need to promote research of aging and aging-related diseases to improve health and longevity of the elderly population. Aging and disease 2015;6:1.

[2] Bianco P, Robey P G. Stem cells in tissue engineering. Nature 2001;414:118-21.

[3] Villasante A, Vunjak-Novakovic G. Tissue-engineered models of human tumors for cancer research. Expert opinion on drug discovery 2015;10:257-68.

[4] Kang X, Xie Y, Powell H M, James Lee L, Belury M A, Lannutti J J, Kniss D A. Adipogenesis of murine embryonic stem cells in a three-dimensional culture system using electro spun polymer scaffolds. Biomaterials 2007;28:450-8.

[5] Nichol J W, Khademhosseini A. Modular tissue engineering: engineering biological tissues from the bottom up. Soft Matter 2009;5:1312-9.

[6] Tan H, Marra K G. Injectable, biodegradable hydrogels for tissue engineering applications. Materials 2010;3:1746-67.

[7] Alexander A, Khan J, Saraf S, Saraf S. Polyethylene glycol (PEG)-Poly (N-isopropylacrylamide) (PNIPAAm) based thermosensitive injectable hydrogels for biomedical applications. European Journal of Pharmaceutics and Biopharmaceutics 2014;88:575-85.

[8] Bakshi A, Fisher O, Dagci T, Himes B T, Fischer I, Lowman A. Mechanically engineered hydrogel scaffolds for axonal growth and angiogenesis after transplantation in spinal cord injury. Journal of Neurosurgery: Spine 2004;1:322-9.

[9] Wang L S, Boulaire J, Chan P P, Chung J E, Kurisawa M. The role of stiffness of gelatin-hydroxyphenylpropionic acid hydrogels formed by enzyme-mediated crosslinking on the differentiation of human mesenchymal stem cell. Biomaterials 2010;31:8608-16.

[10] Benoit D S, Schwartz M P, Durney A R, Anseth K S. Small functional groups for controlled differentiation of hydrogel-encapsulated human mesenchymal stem cells. Nature materials 2008;7:816-23.

[11] Zhao X, Sun X, Yildirimer L, Lang Q, Lin Z Y W, Zheng R, Zhang Y, Cui W, Annabi N, Khademhosseini A. Cell infiltrative hydrogel fibrous scaffolds for accelerated wound healing. Acta Biomater 2017;49:66-77.

[12] Wakitani S, Goto T, Pineda S J, Young R G, Mansour J M, Caplan A I, Goldberg V M. Mesenchymal cell-based repair of large, full-thickness defects of articular cartilage. J Bone Joint Surg Am 1994;76:579-92.

[13] Toh W S, Lee E H, Guo X-M, Chan J K, Yeow C H, Choo A B, Cao T. Cartilage repair using hyaluronan hydrogel-encapsulated human embryonic stem cell-derived chondrogenic cells. Biomaterials 2010;31:6968-80.

[14] Park H, Temenoff J S, Tabata Y, Caplan A I, Mikos A G. Injectable biodegradable hydrogel composites for rabbit marrow mesenchymal stem cell and growth factor delivery for cartilage tissue engineering. Biomaterials 2007;28:3217-27.

[15] Hwang N S, Varghese S, Zhang Z, Elisseeff J. Chondrogenic differentiation of human embryonic stem cell-derived cells in arginine-glycine-aspartate-modified hydrogels. Tissue engineering 2006;12:2695-706.

[16] Bosnakovski D, Mizuno M, Kim G, Takagi S, Okumura M, Fujinaga T. Chondrogenic differentiation of bovine bone marrow mesenchymal stem cells (MSCs) in different hydrogels: influence of collagen type II extracellular matrix on MSC chondrogenesis. Biotechnology and bioengineering 2006;93:1152-63.

[17] Eslahi N, Abdorahim M, Simchi A. Smart Polymeric Hydrogels for Cartilage Tissue Engineering: A Review on the Chemistry and Biological Functions. Biomacromolecules 2016;17:3441-63.

[18] Teo W E, He W, Ramakrishna S. Electrospun scaffold tailored for tissue-specific extracellular matrix. Biotechnology journal 2006;1:918-29.

[19] Nezarati R M, Eifert M B, Cosgriff-Hernandez E. Effects of humidity and solution viscosity on electrospun fiber morphology. Tissue engineering Part C, Methods 2013;19:810-9.

[20] Okuzaki H, Kobayashi K, Yan H. Non-woven fabric of poly(N-isopropylacrylamide) nanofibers fabricated by electrospinning. Synthetic Metals 2009;159:2273-6.

[21] Rockwood D N, Chase D B, Akins R E, Rabolt J F. Characterization of electrospun poly (N-isopropyl acrylamide) fibers. Polymer 2008;49:4025-32.

[22] Comolli N, Neuhuber B, Fischer I, Lowman A. In vitro analysis of PNIPAAm-PEG, a novel, injectable scaffold for spinal cord repair. Acta Biomater 2009;5:1046-55.

[23] Eljarrat-Binstock E, Bentolila A, Kumar N, Harel H, Domb A J. Preparation, characterization, and sterilization of hydrogel sponges for iontophoretic drug-delivery use. Polymers for Advanced Technologies 2007;18:720-30.

[24] Nam J, Huang Y, Agarwal S, Lannutti J. Improved cellular infiltration in electrospun fiber via engineered porosity. Tissue engineering 2007;13:2249-57.

[25] Nam J, Perera P, Rath B, Agarwal S. Dynamic regulation of bone morphogenetic proteins in engineered osteochondral constructs by biomechanical stimulation. Tissue engineering Part A 2013;19:783-92.

[26] Horner C B, Hirota K, Liu J, Maldonado M, Park B H, Nam J. Magnitude-Dependent and Inversely-related Osteogenic/Chondrogenic Differentiation of Human Mesenchymal Stem Cells Under Dynamic Compressive Strain. Journal of tissue engineering and regenerative medicine 2016.

[27] Nam J, Huang Y, Agarwal S, Lannutti J. Materials selection and residual solvent retention in biodegradable electrospun fibers. Journal of Applied Polymer Science 2008;107:1547-54.

[28] Livak K J, Schmittgen T D. Analysis of relative gene expression data using real-time quantitative PCR and the 2(-Delta Delta C(T)) Method. Methods 2001;25:402-8.

[29] Kondiah P P, Tomar L K, Tyagi C, Choonara Y E, Modi G, du Toit L C, Kumar P, Pillay V. A novel pH-sensitive interferon-beta (INF-beta) oral delivery system for application in multiple sclerosis. International journal of pharmaceutics 2013;456:459-72.

[30] Kim H Y, Choi H J. Core-shell structured poly(2-ethylaniline) coated crosslinked poly(methyl methacrylate) nanoparticles by graft polymerization and their electrorheology. RSC Advances 2014;4:28511-8.

[31] Chakraborty P, Bairi P, Roy B, Nandi A K. Rheological and fluorescent properties of riboflavin-poly(N-isopropylacrylamide) hybrid hydrogel with a potentiality of forming Ag nanoparticle. RSC Advances 2014;4:54684-93.

[32] Lee C-F, Zhang G-M, Nieh M-P, Don T-M. Morphology and opto-thermal properties of the thermo-responsive PNIPAAm-protected gold nanorods. Polymer 2016;84:138-47.

[33] Lima AC, Song W, Blanco-Fernandez B, Alvarez-Lorenzo C, Mano J F. Synthesis of temperature-responsive dextran-MA/PNIPAAm particles for controlled drug delivery using superhydrophobic surfaces. Pharmaceutical research 2011;28:1294-305.

[34] Zeng Z, Mo X-m, He C, Morsi Y, El-Hamshary H, El-Newehy M. An in situ forming tissue adhesive based on poly(ethylene glycol)-dimethacrylate and thiolated chitosan through the Michael reaction. Journal of Materials Chemistry B 2016;4:5585-92.

[35] Jadhav S A, Brunella V, Miletto I, Berlier G, Scalarone D. Synthesis of poly(N-isopropylacrylamide) by distillation precipitation polymerization and quantitative grafting on mesoporous silica. Journal of Applied Polymer Science 2016;133:44181.

[36] Cukierman E, Pankov R, Stevens D R, Yamada K M. Taking cell-matrix adhesions to the third dimension. Science 2001;294:1708-12.

[37] Berrier A L, Yamada K M. Cell-matrix adhesion. Journal of cellular physiology 2007;213:565-73.

[38] Grayson W L, Ma T, Bunnell B. Human mesenchymal stem cells tissue development in 3D PET matrices. Biotechnology progress 2004;20:905-12.

[39] Huebsch N, Arany P R, Mao A S, Shvartsman D, Ali O A, Bencherif S A, Rivera-Feliciano J, Mooney D J. Harnessing traction-mediated manipulation of the cell/matrix interface to control stem-cell fate. Nature materials 2010;9:518-26.

[40] Zanetti N C, Solursh M. Induction of chondrogenesis in limb mesenchymal cultures by disruption of the actin cytoskeleton. The Journal of cell biology 1984;99:115-23.

[41] Solursh M, Linsenmayer T F, Jensen K L. Chondrogenesis from single limb mesenchyme cells. Developmental biology 1982;94:259-64.

[42] Haudenschild D R, Chen J, Pang N, Lotz M K, D′Lima D D. Rho kinase-dependent activation of SOX9 in chondrocytes. Arthritis and rheumatism 2010;62:191-200.

[43] Woods A, Wang G, Beier F. RhoA/ROCK signaling regulates Sox9 expression and actin organization during chondrogenesis. The Journal of biological chemistry 2005;280:11626-34.

[44] Anseth K S, Wang C M, Bowman C N. Kinetic evidence of reaction diffusion during the polymerization of multi(meth)acrylate monomers. Macromolecules 1994;27:650-5.

[45] Lee J H, Prud'homme R K, Aksay I A. Cure depth in photopolymerization: Experiments and theory. Journal of Materials Research 2011;16:3536-44.

[46] Liu X Y, Nothias J-M, Scavone A, Garfinkel M, Millis J M. Biocompatibility investigation of polyethylene glycol and alginate-poly-L-lysine for islet encapsulation. ASAIO journal 2010;56:241-5.

[47] Cooperstein M A, Canavan H E. Assessment of cytotoxicity of (N-isopropyl acrylamide) and Poly (N-isopropyl acrylamide)-coated surfaces. Biointerphases 2013;8:19.

[48] Conova L, Vernengo J, Jin Y, Himes B T, Neuhuber B, Fischer I, Lowman A. A pilot study of poly (N-isopropylacrylamide)-g-polyethylene glycol and poly (N-isopropylacrylamide)-g-methylcellulose branched copolymers as injectable scaffolds for local delivery of neurotrophins and cellular transplants into the injured spinal cord: Laboratory investigation. Journal of Neurosurgery: Spine 2011;15:594-604.

[49] Cha C, Jeong J H, Shim J, Kong H. Tuning the dependency between stiffness and permeability of a cell encapsulating hydrogel with hydrophilic pendant chains. Acta Biomater 2011;7:3719-28.

[50] Liu W, Zhan J, Su Y, Wu T, Ramakrishna S, Liao S, Mo X. Injectable hydrogel incorporating with nanoyarn for bone regeneration. Journal of biomaterials science Polymer edition 2014;25:168-80.

[51] Kai D, Prabhakaran M P, Stahl B, Eblenkamp M, Wintermantel E, Ramakrishna S. Mechanical properties and in vitro behavior of nanofiber-hydrogel composites for tissue engineering applications. Nanotechnology 2012;23:095705.

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

All figures, tables, and appendices, as well as publications, patents, and patent applications, cited herein are hereby incorporated by reference in their entirety for all purposes.

What is claimed is:

1. A composite microfiber, comprising an electrospun blend of a thermosensitive hydrogel and a biodegradable polymer.

2. The composite microfiber according to claim 1, wherein the thermosensitive hydrogel is poly(ethylene glycol)-poly(N-isopropylacrylamide) (PEG-PNIPAAm) and wherein the biodegradable polymer is poly(c-caprolactone) (PCL), wherein the PEG-PNIPAAm and PCL are blended throughout the microfiber.

3. The composite microfiber according to claim 1, wherein the biodegradable polymer is natural biodegradable polymer.

4. The composite microfiber according to claim 1, wherein the diameter of the microfiber is in the range of 1 to 50 microns.

5. The composite microfiber according to claim 1, wherein the diameter of the microfiber is in the range of 9 to 13 microns.

6. The composite microfiber according to claim 1, wherein the diameter of the microfiber 11 microns.

7. The composite microfiber according to claim 1, wherein the ratio of thermosensitive hydrogel: biodegradable polymer is 65:35.

8. A scaffold, comprising a plurality of composite microfibers according to claim 1.

9. The scaffold according to claim 8, wherein the scaffold has a thickness of about 2.5 mm.

10. The scaffold according to claim 8, wherein the scaffold further comprises cells cultured within the scaffold.

11. The scaffold according to claim 8, wherein the scaffold has a compressive modulus of between 40-80 kPa.

12. The scaffold according to claim 11, wherein the scaffold has a compressive modulus of about 60 kPa.

13. The scaffold according to claim 10, wherein the scaffold and cells form a shape.

14. A method of making an engineered tissue, the method comprising:
obtaining a plurality of composite microfibers according to claim 1;
seeding cells within the plurality of composite microfibers, while the plurality of composite microfibers is in a dry state and the cells are dispersed in a solution, and
inducing a phase transition of the thermosensitive hydrogel to a gel state upon elevating the temperature of the plurality of composite microfibers and cells, thereby encapsulating the cells in a 3D hydrogel scaffold that forms the engineered tissue.

15. The method according to claim 14, further comprising implanting in a subject the plurality of composite microfibers seeded with the cells, wherein a body temperature of the subject causes the elevating of the temperature of the plurality of composite microfibers and cells, which causes the hydrogel to undergo the phase transition to the gel state.

16. The method according to claim 14, wherein the engineered tissue is a soft tissue, a hard tissue, or a combination of soft and hard tissues selected from the group consisting of bone and cartilage.

17. The method according to claim 14, wherein the cells comprise
mesenchymal stem cells, and wherein the mesenchymal stem cells are cultured within the scaffold under conditions that promote chondrogenic differentiation, thereby producing cartilage tissue.

18. The method according to claim 17, wherein the mesenchymal stem cells are human mesenchymal stem cells (hMSCs).

19. The method according to claim 14, wherein the engineered tissue is a soft tissue.

20. The method according to claim 19, wherein the soft tissue is selected from the group consisting of skin, a vein, an artery and an organ.
